(19) United States
(12) Patent Application Publication
(10) Pub. No.: US 2018/0073057 A1
(43) Pub. Date: Mar. 15, 2018

(54) METHODS FOR CONSTRUCTING CONSECUTIVELY CONNECTED COPIES OF NUCLEIC ACID MOLECULES

(71) Applicant: Dimitra Tsavachidou, Houston, TX (US)

(72) Inventor: Dimitra Tsavachidou, Houston, TX (US)

(21) Appl. No.: 15/817,178

(22) Filed: Nov. 18, 2017

Related U.S. Application Data
(63) Continuation of application No. PCT/US2016/032127, filed on May 13, 2016.
(60) Provisional application No. 62/168,368, filed on May 29, 2015.
(60) Provisional application No. 62/214,777, filed on Sep. 4, 2015.
(60) Provisional application No. 62/243,061, filed on Oct. 17, 2015.

(51) Int. Cl. C12Q 1/68; C12P 19/34

(52) U.S. Cl. CPC C12Q 1/6806; C12P 19/34; C12Q 1/6869

(57) ABSTRACT

Methods for constructing consecutively connected and optionally truncated copies of nucleic acid molecules are disclosed. Consecutively connected copies of nucleic acid molecules can be used to perform sequencing of the same nucleic acid molecules several times, improving overall accuracy of sequencing. Sequencing of truncated copies of nucleic acid molecules can be used to deduce the sequences of nucleic acid molecules from assembling short sequenced segments. Connected copies of nucleic acid molecules can be constructed by first attaching hairpin adaptors to the nucleic acid molecules, and then using strand displacing polymerases to generate complementary strands of the nucleic acid molecule strands connected by the hairpin adaptors.

### PRIORITY CLAIM OF EARLIER APPLICATIONS

This application is a continuation of PCT/US16/32127, filed on May 12, 2016, which claims the benefit of U.S. Provisional Application Ser. No. 62/168,368, filed on May 29, 2015, Ser. No. 62/214,777, filed on Sep. 4, 2015 and Ser. No. 62/243,061, filed on Oct. 17, 2015, which are incorporated by reference herein.

### SEQUENCE LISTING

No sequence listing accompanies this application, because there are no sequences disclosed herein.

### FIELD

The methods provided herein relate to the field of nucleic acid sequencing.

### BACKGROUND

Nucleic acid sequence information is important for scientific research and medical purposes. The sequence information enables medical studies of genetic predisposition to diseases, studies that focus on altered genomes such as the genomes of cancerous tissues, and the rational design of drugs that target diseases. Sequence information is also important for genomic, evolutionary and population studies, genetic engineering applications, and microbial studies of epidemiologic importance. Reliable sequence information is also critical for paternity tests and forensics.

There is a constant need for new technologies that will lower the cost and increase the quality and amount of sequenced output. A promising technology that has the potential to revolutionize sequencing by simplifying the process and lowering the cost is nanopore-based detection. Nanopores are tiny holes that allow DNA translocation through them, which causes detectable disruptions in ionic current according to the sequence of the traversing DNA. Sequencing at single-nucleotide resolution using nanopore devices is performed with reported error rates around 25% (Goodwin et al., 2015). Since these errors occur randomly during sequencing, repeating the sequencing procedure for the same DNA strands several times will generate sequencing results based on consensus derived from replicate readings, thus increasing overall accuracy and reducing overall error rates.

One important drawback of current sequencing technologies is the generation of short sequencing reads. Short sequencing reads provide challenges during their alignment to their corresponding reference genome, thus rendering the retrieval of a properly ordered sequenced genome problematic. The development of technologies that can determine how short sequenced fragments are ordered in their nucleic acid molecule of origin is highly desirable.

### SUMMARY

The methods disclosed herein relate to nucleic acid sequencing. Methods for constructing consecutively connected copies of nucleic acid molecules are disclosed. Methods for constructing consecutively connected and progressively truncated copies of nucleic acid molecules are also disclosed.

Certain embodiments disclosed herein pertain to a method of constructing consecutively connected copies of a nucleic acid molecule comprising two strands, first and second, said method applied to one or more nucleic acid molecules, and said method comprising the steps of: (i) attaching a nucleic acid molecule comprising two strands to an adaptor comprising a nicking endonuclease recognition site, by ligating the 5′ end of the first strand and the 3′ end of the second strand of the nucleic acid molecule to the adaptor; (ii) exposing the nucleic acid molecule and its surroundings to ligases to attach a hairpin adaptor not comprising a nicking endonuclease recognition site, to the 3′ end of the first strand and to the 5′ end of the second strand of the nucleic acid molecule; (iii) exposing the nucleic acid molecule and its surroundings to nicking endonucleases recognizing said nicking endonuclease recognition site, thereby generating a nick with an extendable 3′ end: (a) in the first strand of the nucleic acid molecule whose 5′ end is ligated to the adaptor in step (i), or (b) in a segment of the adaptor ligated to the first strand of the nucleic acid molecule in step (i), or (c) between the adaptor and the first strand of the nucleic acid molecule whose 5′ end is ligated to the adaptor in step (i); (iv) extending said extendable 3′ end by using polymerase molecules with strand displacing activity; and (v) repeating steps (ii) through (iv) at least once, thereby allowing consecutive construction of copies of the nucleic acid molecule connected to one another.

In some related embodiments, all steps are conducted in the same reaction solution comprising nicking endonucleases, polymerases and ligases. In some other related embodiments, the reaction solution participates in temperature cycles comprising a temperature setting that favors nicking and extension and another temperature setting that favors ligation. Still further, in other related embodiments, reagents used for at least two steps are included in a single reaction solution.

Certain embodiments disclosed herein pertain to a method of constructing consecutively connected copies of a nucleic acid molecule comprising two strands, said method applied to one or more nucleic acid molecules, and said method comprising the steps of: (i) Ligating hairpin adaptors to a nucleic acid molecule, said hairpin adaptors comprising nicking endonuclease recognition sites; (ii) Generating nicks with extendable 3′ ends within the nicking endonuclease recognition sites of said hairpin adaptors, by exposing the nucleic acid molecule and its surroundings to nicking endonucleases; (iii) extending said extendable 3′ ends by using polymerase molecules with strand displacing activity, thereby generating hairpin constructs; (iv) ligating hairpin adaptors to the hairpin constructs in step (iii), said hairpin adaptors not comprising said nicking endonuclease recognition sites, thereby generating circularized constructs comprising a single nicking endonuclease recognition site each; (v) generating nicks with extendable 3′ ends within the nicking endonuclease recognition sites, by exposing to nicking endonucleases; (vi) extending the 3′ ends of the nicks in step (v) by using strand-displacing polymerases; (vii) repeating nick formation and extension, producing displaced single strands that can form hairpins with extendable 3′ ends; (viii) extending the extendable 3′ ends from step (vii), thus producing long hairpin constructs; (ix) ligating hairpin adaptors to the long hairpin constructs in step (viii), said hairpin adaptors regenerating nicking endonuclease recognition sites upon ligation; and (x) repeating steps (v) through (ix).

### BRIEF DESCRIPTION OF THE DRAWINGS

In the detailed description of various embodiments usable within the scope of the present disclosure, presented below, reference is made to the accompanying drawings, in which:

FIG. 1 is a schematic diagram of a method for constructing a copy of a DNA molecule, said copy being connected to said DNA molecule;

FIGS. 2A through 2E are schematic diagrams of a method for constructing truncated copies of a DNA molecule;

FIG. 3 is a schematic diagram of a method for preparing DNA copies for sequencing;

FIG. 4 is a schematic diagram of a method for preparing DNA copies attached to identifiers for sequencing;

FIG. 5 is a schematic diagram of two hairpin adaptors;

FIG. 6 is a schematic diagram of a hairpin adaptor;

FIG. 7 is a schematic diagram of a hairpin adaptor;

FIG. 8 is a schematic diagram of a method for preparing a DNA copy for single-read sequencing;

FIGS. 9A through 9C are schematic diagrams of a method for constructing truncated copies of a DNA molecule;

FIG. 10 is a schematic diagram of a method for sequencing progressively shortened copies of a DNA molecule;

FIGS. 11A and 11B are schematic diagrams of two methods for preparing rolling-circle amplification products for sequencing;

FIGS. 12A and 12B are schematic diagrams of a method for constructing truncated copies of a DNA molecule;

FIGS. 13A through 13C are schematic diagrams of a method for constructing truncated copies of a DNA molecule; and

FIGS. 14A through 14C are schematic diagrams of a method for constructing copies of a nucleic acid molecule.

### DETAILED DESCRIPTION

Methods described herein construct copies of a nucleic acid molecule that are consecutively connected to the nucleic acid molecule. Such copies are useful because they can be sequenced consecutively by a sequencer such as a nanopore device, enabling replicate readings, thus improving overall sequencing accuracy.

Other methods described herein construct copies of a nucleic acid molecule that are consecutively connected to the nucleic acid molecule, and progressively truncated. Such copies can be released, for example, by using restriction enzymes, then attached to adaptors, then optionally amplified and sequenced. Such copies can be attached to “origin identifiers” that can reveal their relationship to their nucleic acid molecule of origin. Such copies can also be attached to “copy identifiers” that can reveal the order with which such copies are connected to the nucleic acid molecule during copy construction. Such progressively truncated copies are useful because they can be sequenced, along with their associated origin and copy identifiers, using short-read sequencing technologies, and can be aligned to their reference genome in the proper order, according to the information stored in the sequences of their associated origin and copy identifiers.

We show the particulars herein by way of example and for purposes of illustrative discussion of the embodiments. We present these particulars to provide what we believe to be the most useful and readily understood description of the principles and conceptual aspects of various embodiments of the disclosure. In this regard, we make no attempt to show structural details in more detail than is necessary for the fundamental understanding of the disclosed methods. We intend that the description should be taken with the drawings. This should make apparent to those skilled in the art how the several forms of the disclosed methods are embodied in practice.

### Terms and Definitions

We mean and intend that the following definitions and explanations are controlling in any future construction unless clearly and unambiguously modified in the following examples or when application of the meaning renders any construction meaningless or essentially meaningless. In cases where the construction of the term would render it meaningless or essentially meaningless, we intend that the definition should be taken from Webster's Dictionary 3rd Edition.

“Nucleotide” as used herein refers to a phosphate ester of a nucleoside, e.g., a mono-, or a triphosphate ester. A nucleoside is a compound consisting of a purine, deazapurine, or pyrimidine nucleoside base, e.g., adenine, guanine, cytosine, uracil, thymine, 7-deazaadenine, that can be linked to the anomeric carbon of a pentose sugar, such a ribose, 2′-deoxyribose, or 2′, 3′-di-deoxyribose. The most common site of esterification is the hydroxyl group connected to the C-5 position of the pentose (also referred to herein as 5′ position or 5′ end). The C-3 position of the pentose is also referred to herein as 3′ position or 3′ end. The term “deoxyribonucleotide” refers to nucleotides with the pentose sugar 2′-deoxyribose. The term “ribonucleotide” refers to nucleotides with the pentose sugar ribose. The term “dideoxyribonucleotide” refers to nucleotides with the pentose sugar 2′, 3′-di-deoxyribose.

A nucleotide may be incorporated and/or modified, in the event that it is stated as such, or implied or allowed by context.

“Complementary” generally refers to specific nucleotide duplexing to form canonical Watson-Crick base pairs, as is understood by those skilled in the art. For example, two nucleic acid strands or parts of two nucleic acid strands are said to be complementary or to have complementary sequences in the event that they can form a perfect base-paired double helix with each other.

“Nucleic acid molecule” is a polymer of nucleotides consisting of at least two nucleotides covalently linked together. A nucleic acid molecule can be a polynucleotide or an oligonucleotide. A nucleic acid molecule can be deoxyribonucleic acid (DNA), ribonucleic acid (RNA), or a combination of both. A nucleic acid molecule may comprise methylated nucleotides generated in vivo or by treating with methyltransferases (e.g., dam methyltransferase). A nucleic acid molecule may be single stranded or double stranded, as specified. A double stranded nucleic acid molecule may comprise non-complementary segments.

Nucleic acid molecules generally comprise phosphodiester bonds, although in some cases, they may have alternate backbones, comprising, for example, phosphoramide ((Beaucage and Iyer, 1993) and references therein; (Letsinger and Mungall, 1970); (Sprinzl et al., 1977); (Letsinger et al., 1986); (Sawai, 1984); and (Letsinger et al., 1988)), phosphorothioate ((Mag et al., 1991); and U.S. Pat. No. 5,644,048 (Yau, 1997)), phosphorodithioate (Brill et al., 1989), O-methylphosphoroamidite linkages (Eckstein, 1992), and peptide nucleic acid backbones and linkages ((Egholm et al., 1992); (Meier and Engels, 1992); (Egholm et al., 1993); and (Carlsson et al., 1996)). Other analog nucleic acids include those with bicyclic structures including locked nucleic acids, (Koshkin et al., 1998); positive backbones (Dempcy et al., 1995); non-ionic backbones (U.S. Pat. No. 5,386,023 (Cook and Sanghvi, 1992), U.S. Pat. No. 5,637,684 (Cook et al., 1997), U.S. Pat. No. 5,602,240 (Mesmaeker et al., 1997), U.S. Pat. No. 5,216,141 (Benner, 1993) and U.S. Pat. No. 4,469,863 (Ts'o and Miller, 1984); (von Kiedrowski et al., 1991); (Letsinger et al., 1988); (Jung et al., 1994); (Sanghvi and Cook, 1994); (De Mesmaeker et al., 1994); (Gao and Jeffs, 1994); (Horn et al., 1996)) and non-ribose backbones, including those described in U.S. Pat. No. 5,235,033 (Summerton et al., 1993) and U.S. Pat. No. 5,034,506 (Summerton and Weller, 1991), and (Sanghvi and Cook, 1994). Nucleic acids containing one or more carbocyclic sugars are also included within the definition of nucleic acids (Jenkins and Turner, 1995). Several nucleic acid analogs are described in Rawls, C & E News Jun. 2, 1997 page 35 (RAWLS, 1997).

All methods described herein to be performed on “a nucleic acid molecule”, can be applied to a single nucleic acid molecule, or more than one nucleic acid molecules. For example, said methods can apply to many identical nucleic acid molecules, such as PCR copies derived from a single nucleic acid molecule. In another example, said methods can also apply to many nucleic acid molecules of diverse sequences, such as extracted and sheared fragments of genomic DNA molecules. In another example, said methods can also apply to a plurality of groups of nucleic acid molecules, each group comprising copies of a specific nucleic acid molecule, such as the combination of products derived from multiple PCR assays. Examples mentioned above are non-limiting.

A nucleic acid molecule may be linked to a surface (e.g., functionalized solid support, adaptor-coated beads, primer-coated surfaces, etc.).

Unless stated otherwise, a “nucleic acid molecule” that participates in reactions, or is said to be exposed to conditions or subjected to processes (or other equivalent phrase) to cause a reaction or event to occur, comprises the nucleic acid molecule and everything associated with it (sometimes referred to as “parts” or “surroundings”). Incorporated nucleotides, attached adaptors, hybridized primers or strands, etc., that are associated (e.g., bound, hybridized, attached, incorporated, ligated, etc.) with the nucleic acid molecule prior to or during a method described herein, are or become part of the nucleic acid molecule, and are comprised in the term “nucleic acid molecule”. For example, a nucleotide that is incorporated into the nucleic acid molecule in a step becomes part of the nucleic acid molecule in the next steps. For example, an adaptor that is already attached to the nucleic acid molecule prior to being subjected to methods described herein, is part of the nucleic acid molecule.

The term “adaptor” refers to an oligonucleotide or polynucleotide, single-stranded (e.g., hairpin adaptor) or double-stranded, comprising at least a part of known sequence. Adaptors may include no sites, or one or more sites for restriction endonuclease recognition, or recognition and cutting. Adaptors may comprise methyltransferase recognition sites. Adaptors may comprise one or more cleavable features or other modifications. Adaptors may or may not be anchored to a surface, and may comprise one or more modifications (for example, to allow anchoring to lipid membranes or other surfaces) and/or be linked to one or more enzymes (e.g. helicases) or other molecules.

A “hairpin adaptor” is an adaptor comprising a single strand with at least a part exhibiting self-complementarity. Such self-complementarity forms a double-stranded structure. Hairpin adaptors may comprise modified nucleotides or other modifications that, for example, enable attachment to surfaces, nicking, restriction enzyme recognition, etc.

The term “polymerization” refers to the process of covalently connecting nucleotides to form a nucleic acid molecule (or a nucleic acid construct), or covalently connecting nucleotides via backbone bonds, one nucleotide at a time, to an existing nucleic acid molecule or a nucleic acid construct. The latter case is also termed “extension by polymerization”. Polymerization (extension by polymerization) can be template-dependent or template-independent. In template-dependent polymerization, the produced strand is complementary to another strand which serves as a template during the polymerization reaction, whereas in template-independent polymerization, addition of nucleotides to a strand does not depend on complementarity.

“Template strand”: As known by those skilled in the art, the term “template strand” refers to the strand of a nucleic acid molecule that serves as a guide for nucleotide incorporation into the nucleic acid molecule comprising an extendable 3′ end, in the event that the nucleic acid molecule is subjected to a template-dependent polymerization reaction. The template strand guides nucleotide incorporation via base-pair complementarity, so that the newly formed strand is complementary to the template strand.

“Extendable 3′ end” refers to a free 3′ end of a nucleic acid molecule or nucleic acid construct, said 3′ end being capable of forming a backbone bond with a nucleotide during template-dependent polymerization. “Extendable strand” is a strand of a nucleic acid molecule that comprises an extendable 3′ end.

A “construct” may refer to adaptors (hairpins or others) or other method-made entities.

“Segment”: When referring to nucleic acid molecules, or nucleic acid constructs, “segment” is a part of a nucleic acid molecule (e.g., template strand) or a nucleic acid construct (e.g., adaptor) comprising at least one nucleotide.

The terms “attachment” and “ligation” are used interchangeably, unless otherwise stated or implied by context.

When referring to restriction enzymes, including nicking endonucleases, the terms “recognition site” and “restriction site” are used interchangeably, unless otherwise stated or implied by context, and refer to sites that can be recognized by such enzymes which may cut inside or outside of these sites.

A “mismatch” may be a single-base mismatch or a more-than-one-base mismatch. It may refer to a substitution, or insertion or deletion or combinations thereof.

An “identifier” refers to a sequence that comprises information about a nucleic acid molecule and/or a copy of a nucleic acid molecule. For example, an identifier may be an origin identifier or a copy identifier, as described below. Identifier sequences may be known in advanced, or constructed randomly and determined by sequencing. Generating random sequences is well known to those skilled in the art, as for example in the case of constructing random oligonucleotides to be used as primers.

The term “origin identifier” refers to a sequence which can identify whether one or more copies are copies of a specific nucleic acid molecule that the origin identifier represents.

The term “copy identifier” refers to a sequence which can identify a specific full-length or truncated copy of a nucleic acid molecule, or can reveal: (i) whether a copy of a nucleic acid molecule is full-length or truncated, and (ii) which round of truncation created the truncated copy.

### Nucleic Acid Molecules

Nucleic acid molecules can be obtained from several sources using extraction methods known in the art. Examples of sources include, but are not limited to, bodily fluids (such as blood, urine, serum, lymph, saliva, anal and vaginal secretions, perspiration and semen) and tissues (normal or pathological such as tumors) of any organism, including human samples; environmental samples (including, but not limited to, air, agricultural, water and soil samples); research samples (such as PCR products); purified samples, such as purified genomic DNA, RNA, etc. In certain embodiments, genomic DNA is obtained from whole blood or cell preparations from blood or cell cultures. In further embodiments, nucleic acid molecules comprise a subset of whole genomic DNA enriched for transcribed sequences. In further embodiments, the nucleic acid molecules comprise a transcriptome (i.e., the set of mRNA or “transcripts” produced in a cell or population of cells) or a methylome (i.e., the population of methylated sites and the pattern of methylation in a genome).

In some embodiments, nucleic acid molecules of interest are genomic DNA molecules. Nucleic acid molecules can be naturally occurring or genetically altered or synthetically prepared.

Nucleic acid molecules can be directly isolated without amplification, or isolated by amplification using methods known in the art, including without limitation polymerase chain reaction (PCR), strand displacement amplification (SDA), multiple displacement amplification (MDA), rolling circle amplification (RCA), rolling circle amplification (RCR) and other amplification methodologies. Nucleic acid molecules may also be obtained through cloning, including but not limited to cloning into vehicles such as plasmids, yeast, and bacterial artificial chromosomes.

In some embodiments, the nucleic acid molecules are mRNAs or cDNAs. Isolated mRNA may be reverse transcribed into cDNAs using conventional techniques, as described in Genome Analysis: A Laboratory Manual Series (Vols. I-IV) (Green, 1997) or Molecular Cloning: A Laboratory Manual (Green and Sambrook, 2012).

Genomic DNA is isolated using conventional techniques, for example as disclosed in Molecular Cloning: A Laboratory Manual (Green and Sambrook, 2012). The genomic DNA is then fractionated or fragmented to a desired size by conventional techniques including enzymatic digestion using restriction endonucleases, random enzymatic digestion, or other methods such as shearing or sonication.

Fragment sizes of nucleic acid molecules can vary depending on the source and the library construction methods used. In some embodiments, the fragments are 300 to 600 or 200 to 2000 nucleotides or base pairs in length. In other embodiments, the fragments are less than 200 nucleotides or base pairs in length. In other embodiments, the fragments are more than 2000 nucleotides or base pairs in length.

In a further embodiment, fragments of a particular size or in a particular range of sizes are isolated. Such methods are well known in the art. For example, gel fractionation can be used to produce a population of fragments of a particular size within a range of base pairs, for example for 500 base pairs±50 base pairs.

In one embodiment, the DNA is denatured after fragmentation to produce single stranded fragments.

In one embodiment, an amplification step can be applied to the population of fragmented nucleic acid molecules. Such amplification methods are well known in the art and include without limitation: polymerase chain reaction (PCR), ligation chain reaction (sometimes referred to as oligonucleotide ligase amplification OLA), cycling probe technology (CPT), strand displacement assay (SDA), transcription mediated amplification (TMA), nucleic acid sequence based amplification (NASBA), rolling circle amplification (RCA) (for circularized fragments), and invasive cleavage technology.

In some embodiments, a controlled random enzymatic (“CoRE”) fragmentation method is utilized to prepare fragments (Peters et al., 2012).

Other suitable enzymatic, chemical or photochemical cleavage reactions that may be used to cleave nucleic acid molecules include, but not limited to, those described in WO 07/010251 (Barnes et al., 2007) and U.S. Pat. No. 7,754,429 (Rigatti and Ost, 2010), the contents of which are incorporated herein by reference in their entirety.

In some cases, particularly when it is desired to isolate long fragments (such as fragments from about 150 to about 750 kilobases in length), DNA isolation methods described in U.S. Pat. No. 8,518,640 (Drmanac and Callow, 2013) can be applied.

### Processing and Anchoring of Nucleic Acid Molecules

In some embodiments, the nucleic acid molecules are anchored to the surface of a substrate. Examples of relevant methods are described in U.S. Pat. No. 7,981,604 (Quake, 2011), U.S. Pat. No. 7,767,400 (Harris, 2010), U.S. Pat. No. 7,754,429 (Rigatti and Ost, 2010), U.S. Pat. No. 7,741,463 (Gormley et al., 2010) and WO 2010048386 A1 (Pierceall et al., 2010), included by reference herein in their entirety. The substrate can be a solid support (e.g., glass, quartz, silica, polycarbonate, polypropylene or plastic), a semi-solid support (e.g., a gel or other matrix), a porous support (e.g., a nylon membrane or cellulose) or combinations thereof or any other conventionally non-reactive material. Suitable substrates of various shapes include, for example, planar supports, spheres, microparticles, beads, membranes, slides, plates, micromachined chips, tubes (e.g., capillary tubes), microwells, microfluidic devices, channels, filters, or any other structure suitable for anchoring a nucleic acid molecule. Substrates can include planar arrays or matrices capable of having regions that include populations of nucleic acid molecules or primers. Examples include nucleoside-derivatized CPG and polystyrene slides; derivatized magnetic slides; polystyrene grafted with polyethylene glycol, and the like.

In some embodiments, the substrate is selected to not create significant noise or background for fluorescent detection methods. In certain embodiments, the substrate surface to which nucleic acid molecules are anchored can also be the internal surface of a flow cell in a microfluidic apparatus, e.g., a microfabricated synthesis channel. By anchoring the nucleic acid molecules, unincorporated nucleotides can be removed from the synthesis channels by a washing step.

In one embodiment, a substrate is coated to allow optimum optical processing and nucleic acid molecule anchoring. Substrates can also be treated to reduce background. Exemplary coatings include epoxides, and derivatized epoxides (e.g., with a binding molecule, such as streptavidin).

In some embodiments, the nucleic acid molecules are anchored to a surface prior to hybridization to primers or ligation to adaptors. In certain embodiments, the nucleic acid molecules are hybridized to primers first or ligated to adaptors first and then anchored to the surface. In still some embodiments, primers (or adaptors) are anchored to a surface, and nucleic acid molecules hybridize to the primers or attach to the adaptors. In some embodiments, the primer is hybridized to the nucleic acid molecule prior to providing nucleotides for the polymerization reaction. In some, the primer is hybridized to the nucleic acid molecule while the nucleotides are being provided. In still some embodiments, the polymerizing agent is anchored to the surface.

Various methods can be used to anchor or immobilize the nucleic acid molecules or the primers or the adaptors to the surface of the substrate, such as, the surface of the synthesis channels or reaction chambers. The immobilization can be achieved through direct or indirect bonding to the surface. The bonding can be by covalent linkage (Joos et al., 1997); (Oroskar et al., 1996); and (Khandjian, 1986). The bonding can also be through non-covalent linkage. For example, Biotin-streptavidin (Taylor et al., 1991) and digoxigenin with anti-digoxigenin (Smith et al., 1992) are commonly used for anchoring polynucleotides to surfaces and parallels. Alternatively, the anchoring can be achieved by anchoring a hydrophobic chain into a lipid monolayer or bilayer. Other methods for anchoring nucleic acid molecules to supports can also be used.

While diverse nucleic acid molecules can be each anchored to and processed in a separate substrate or in a separate synthesis channel, multiple nucleic acid molecules can also be analyzed on a single substrate (e.g. in a single microfluidic channel). In the latter case, the nucleic acid molecules can be bound to different locations on the substrate (e.g. at different locations along the flow path of the channel). This can be accomplished by a variety of different methods known in the art.

Methods of creating surfaces with arrays of oligonucleotides have been described, e.g., in U.S. Pat. No. 5,744,305 (Fodor et al., 1998), U.S. Pat. No. 5,837,832 (Chee et al., 1998), and U.S. Pat. No. 6,077,674 (Schleifer and Tom-Moy, 2000).

Another method for anchoring multiple nucleic acid molecules to the surface of a single substrate (e.g. in a single channel) is to sequentially activate portions of the substrate and anchor nucleic acid molecules to them. Activation of the substrate can be achieved by either optical or electrical methods, as described in U.S. Pat. No. 7,981,604 (Quake, 2011), which is incorporated herein by reference in its entirety.

In certain embodiments, different nucleic acid molecules can also be anchored to the surface randomly as the reading of each individual molecule may be analyzed independently from the others. Any other known methods for anchoring nucleic acid molecules may be used.

In some embodiments, the nucleic acid molecules are ligated to adaptors. Relevant methods are described in U.S. Pat. No. 7,741,463 (Gormley et al., 2010) and U.S. Pat. No. 7,754,429 (Rigatti and Ost, 2010), whose contents are incorporated herein by reference in their entirety. Adaptors can be ligated to nucleic acid molecules prior to anchoring to the solid support, or they may be anchored to the solid support prior to ligation to the nucleic acid molecule. The adaptors are typically oligonucleotides or polynucleotides (double stranded or single stranded) that may be synthesized by conventional methods. In some embodiments, adaptors have a length of about 10 to about 250 nucleotides. In certain embodiments, adaptors have a length of about 50 nucleotides. The adaptors may be connected to the 5′ and 3′ ends of nucleic acid molecules by a variety of methods (e.g. subcloning, ligation, etc).

In order to initiate construction of a copy of a nucleic acid molecule, an extendable 3′ end is formed in the nucleic acid molecule, or in an adaptor ligated to the nucleic acid molecule. One way is to denature the nucleic acid molecule linked to the adaptor and hybridize a primer that is complementary to a specific sequence within the adaptor. Another way is to create a nick in the nucleic acid molecule by using a restriction endonuclease that recognizes a specific sequence within the adaptor and cleaves only one of the strands. This can be accomplished, for example, by using a nicking endonuclease that has a non-palindromic recognition site. Suitable nicking endonucleases are known in the art. Nicking endonucleases are available, for example from New England BioLabs. Suitable nicking endonucleases are also described in (Walker et al., 1992); (Wang and Hays, 2000); (Higgins et al., 2001); (Morgan et al., 2000); (Xu et al., 2001); (Heiter et al., 2005); (Samuelson et al., 2004); and (Zhu et al., 2004), which are incorporated herein by reference in their entirety for all purposes. Additional methods and details can be found in U.S. Pat. No. 8,518,640 (Drmanac and Callow, 2013) and US 2013/0327644 (Turner and Korlach, 2013) which are included herein by reference in their entirety.

In another embodiment, the nucleic acid molecule is subject to a 3′-end tailing reaction. Example of this method is described in WO 2010/048386 A1 (Pierceall et al., 2010), which is referenced herein in its entirety. A poly-A tail is generated on the free 3′-OH of the nucleic acid molecule. The tail may be enzymatically generated using terminal deoxynucleotidyl transferase (TdT) and dATP. Typically, a poly-A tail containing 50 to 70 adenine-containing nucleotides is constructed. The poly-A tail facilitates hybridization of the nucleic acid molecule to poly-dT primer molecules anchored to a surface. In principle, nucleic acid molecule tailing can be carried out with a variety of dNTPs (or heterogeneous combinations), e.g., dATP. dATP can be used because TdT adds dATP with predictable kinetics useful to synthesize a 50-70 nucleotide tail. Similarly, RNA may be labeled with poly-A polymerase enzyme and ATP.

In some embodiments, the nucleic acid molecules are processed individually, as single molecules. In one embodiment, a single nucleic acid molecule is anchored to a solid surface and processed. In another embodiment, various nucleic acid molecules are anchored on a solid surface in conditions that allow individual single molecule processing. Examples of nucleic acid molecule concentrations and conditions allowing single molecule processing of multiple nucleic acid molecules are given in U.S. Pat. No. 7,767,400 (Harris, 2010). In another embodiment, one nucleic acid molecule is first amplified and then some of its copies are processed. In another embodiment, some nucleic acid molecules that are copies of the same nucleic acid molecule are amplified and processed. In another embodiment, various single nucleic acid molecules are first amplified forming distinct colonies or clusters and then processed simultaneously. Examples are described in U.S. Pat. No. 8,476,044 (Mayer et al., 2013) and US 2012/0270740 (Edwards, 2012), which are included herein as references in their entirety.

In some embodiments, nucleic acid molecules are anchored to surfaces that can be exposed to various reagents and washed in an automated manner. In other embodiments, nucleic acid molecules are anchored to surfaces that are housed in a flow chamber of a microfluidic device having an inlet and outlet to allow for renewal of reactants which flow past the immobilized moieties. Examples are described in U.S. Pat. No. 7,981,604 (Quake, 2011), U.S. Pat. No. 6,746,851 (Tseung et al., 2004), US 2013/0260372 (Buermann et al., 2013), and US 2013/0184162 (Bridgham et al., 2013), which are included herein as references in their entirety.

The methods described herein can apply to a single nucleic acid molecule or to more than one nucleic acid molecules. Methods to capture and handle individual nucleic acid molecules are known in the art. For examples, dilution methods are known that allow the presence of a single nucleic acid molecule inside a well, a microwell, a tube, a microtube, a nanowell, etc. Several methods are known that allow binding of a single nucleic acid molecule on a bead, on a well surface, etc. Methods are also known that allow single nucleic acid molecules to be linked onto a surface at a distance from other single nucleic acid molecules. Representative references describing methods using single nucleic acid molecules are the following: (Shuga et al., 2013); (Thompson and Steinmann, 2010); (Efcavitch and Thompson, 2010); (Hart et al., 2010); (Chiu et al., 2009); (Ben Yehezkel et al., 2008); (Metzker, 2010).

### Restriction Enzymes and Exonucleases

In many embodiments, nicking endonucleases are used to generate an extendable 3′ end within a nucleic acid molecule, or adaptor, etc. A nicking endonuclease can hydrolyze only one strand of a duplex to produce DNA molecules that are “nicked” rather than cleaved. The nicking can result in a 3′-hydroxyl and a 5′-phosphate. Examples of nicking enzymes include but are not limited to Nt.CviPII, Nb.BsmI, Nb.BbvCI, Nb.BsrDI, Nb.BtsI, Nt.BsmAI, Nt.BspQI, Nt.AlwI, Nt.BbvCI, or Nt.BstNBI. Nicking endonucleases may have non-palindromic recognition sites. Nicking endonucleases are available, for example from New England BioLabs. Suitable nicking endonucleases are also described in (Walker et al., 1992); (Wang and Hays, 2000); (Higgins et al., 2001); (Morgan et al., 2000); (Xu et al., 2001); (Heiter et al., 2005); (Samuelson et al., 2004); and (Zhu et al., 2004), which are incorporated herein by reference in their entirety for all purposes.

In several embodiments, copies of a nucleic acid molecule are truncated. Truncation can be done by using restriction endonucleases that can cut into a region of unknown sequence, said region being located away from their recognition site. Enzymes such as MmeI or EcoP15 can be used. EcoP15I is a type III restriction enzyme that recognizes the sequence motif CAGCAG and cleaves the double stranded DNA molecule 27 base pairs downstream of the CAGCAG motif. The cut site contains a 2 base 5′-overhang that can be end repaired to give a 27 base blunt ended duplex. Under normal in vivo conditions EcoP15I requires two CAGCAG motifs oriented in a head to head orientation on opposite strands of the double stranded molecule, and then the enzyme cleaves the duplex at only one of the two sites. However, under specific in vitro conditions in the presence of the antibiotic compound sinefungin (Sigma cat number S8559) EcoP15I has the desired effect of inducing cleavage of a double stranded duplex at all CAGCAG sequences present in a sequence irrespective of number or orientation (Raghavendra and Rao, 2005).

In several embodiments, hairpin and other adaptors may comprise one or more restriction enzyme binding sites and or cleavage sites. Examples of restriction enzymes include, but are not limited to: AatII, Acc65I, AccI, AciI, AclI, AcuI, AfeI, AflII, AflIII, AgeI, AhdI, AleI, AluI, AlwI, AlwNI, ApaI, ApaLI, ApeKI, ApoI, AscI, AseI, AsiSI, AvaI, AvaIl, AvrII, BaeGI, BaeI, BamHI, BanI, BanII, BbsI, BbvCI, BbvI, BccI, BceAI, BcgI, BciVI, BelI, BfaI, BfuAI, BfuCI, BglI, BglII, BlpI, BmgBI, BmrI, BmtI, BpmI, Bpu10I, BpuEI, BsaAI, BsaBI, BsaHI, BsaI, BsaJI, BsaWI, BsaXI, BseRI, BseYI, BsgI, BsiEI, BsiHKAI, BsiWI, BslI, BsmAI, BsmBI, BsmFI, BsmI, BsoBI, Bsp1286I, BspCNI, BspDI, BspEI, BspHI, BspMI, BspQI, BsrBI, BsrDI, BsrFI, BsrGI, BsrI, BssHII, BssKI, BssSI, BstAPI, BstBI, BstEII, BstNI, BstUI, BstXI, BstYI, BstZ17I, Bsu36I, BtgI, BtgZI, BtsCI, BtsI, Cac8I, ClaI, CspCI, CviAII, CviKI-1, CviQI, DdeI, DpnI, DpnII, DraI, DraIII, DrdI, EaeI, EagI, EarI, EciI, Eco53kI, EcoNI, EcoO109I, EcoP15I, EcoRI, EcoRV, FatI, FauI, Fnu4HI, FokI, FseI, FspI, HaeII, HaeIII, HgaI, HhaI, HincII, HindIII, HinfI, HinP11, HpaI, HpaII, HphI, Hpyl66I, Hpyl88I, Hpyl88 III, Hpy99I, HpyAV, HpyCH4III, HpyCH4IV, HpyCH4V, KasI, KpnI, MboI, MboII, MfeI, MluI, MlyI, MmeU, MnlI, MscI, MseI, MslI, MspAlI, MspI, MwoI, NaeI, NarI, Nb.BbvCI, Nb.BsmI, Nb.BsrDI, Nb.BtsI, NciI, NcoI, NdeI, NgoMIV, NheI, NlaIII, NIaIV, NmeAIII, NotI, NruI, NsiI, NspI, Nt.AlwI, Nt.BbvCI, Nt.BsmAI, Nt.BspQI, Nt.BstNBI, Nt.CviPII, PacI, PaeR7I, PciI, PflFI, PflMI, PhoI, PleI, PmeI, PmlI, PpuMI, PshAI, PsiI, PspGI, PspOMI, PspXI, PstI, PvuI, PvuII, RsaI, RsrII, SacI, SacII, SalI, SapI, Sau3AI, Sau96I, SbfI, ScaI, ScrFI, SexAI, SfaNI, SfcI, SfiI, SfoI, SgrAI, SmaI, SmlI, SnaBI, SpeI, SphI, SspI, StuI, StyD4I, StyI, SwaI, T, Taq.alpha.I, TfiI, TliI, TseI, Tsp45I, Tsp5091, TspMI, TspRI, Tth11I, XbaI, XcmI, XhoI, XmaI, XmnI, or ZraI.

Restriction enzymes used in some embodiments may be Type IIS restriction enzymes, which can cleave DNA at a defined distance from a non-palindromic asymmetric recognition site. Non-limiting examples of Type IIS restriction enzymes include AarI, Acc36I, AccBSI, AciI, AclWI, AcuI, AloI, Alw26I, AlwI, AsuHPI, BaeI, BbsI, BbvCI, BbvI, BccI, BceAI, BcgI, BciVI, BfiI, BfuAI, BfuI, BmgBI, BmrI, BpiI, BpmI, Bpu10I, Bpu10I, BpuAI, BpuEI, BsaI, BsaMI, BsaXI, Bse1I, Bse3DI, BseGI, BseMI, BseMII, BseNI, BseRI, BseXI, BseYI, BsgI, BsmAI, BsmBI, BsmFI, BsmI, Bso31I, BspCNI, BspMI, BspQI, BspTNI, BsrBI, BsrDI, BsrI, BsrSI, BssSI, Bst2BI, Bst6I, BstF5I, BstMAI, BstV1I, BstV2I, BtgZI, BtrI, BtsCI, BtsI, CspCI, Eam1104I, EarI, EciI, Eco31I, Eco57I, Eco57MI, Esp3I, FauI, FauI, FokI, GsuI, HgaI, Hin4I, HphI, HpyAV, Ksp632I, LweI, MbiI, MboII, MlyI, MmeI, MnlI, Mva12691, NmeAIII, PctI, PleI, PpiI, PpsI, PsrI, SapI, SchI, SfaNI, SmuI, TspDTI, TspGWI, or Taq II. A restriction enzyme can bind recognition sequence within an adaptor and cleave sequence outside the adaptor.

The restriction enzyme can be a methylation sensitive restriction enzyme. The methylation sensitive restriction enzyme can specifically cleave methylated DNA. The methylation sensitive restriction enzyme can specifically cleave unmethylated DNA. A methylation sensitive enzyme can include, e.g., DpnI, Acc65I, KpnI, ApaI, Bsp120I, Bsp143I, MboI, BspOI, NheI, Cfr9I, SmaI, Csp6I, RsaI, Ec1136II, SacI, EcoRII, MvaI, HpalI, MSpJI, LpnPI, FsnEI, DpnII, McrBc, or MspI.

In some embodiments, 3′-to-5′ exonucleases such as exonuclease III can be used to truncate the 3′ end of a copy of a nucleic acid molecule. In a subsequent step, 5′-to-3′ exonucleases such as RecJf, or endonucleases that specifically remove single strands, such as mung bean nuclease, can be used to remove the remaining single-stranded segment of the copy. The level of truncation can be modulated as described previously for partial digestion protocols using exonuclease III (Guo and Wu, 1982).

In some embodiments, 5′-to-3′ exonucleases such as T7 exonuclease are used. In a subsequent step, 3′-to-5′ exonucleases such as exonuclease I or T, or endonucleases that specifically remove single strands, such as mung bean nuclease, can be used to remove the remaining single-stranded segment of the copy.

### Methyltransferases

In many embodiments, methyltransferases are used to methylate nucleic acid molecules and their copies, hairpin adaptors, other types of adaptors or other constructs, in order to protect them from restriction enzyme cutting. Methylation may occur within a restriction endonuclease site or near a restriction endonuclease site, and have a blocking effect.

DNA methyltransferases transfer a methyl group from S-adenosylmethionine (SAM) to a nucleotide base such as cytosine or adenine, and can be used to methylate DNA at specific sites. DNA methyltransferases were originally discovered as parts of restriction-modification (R-M) systems wherein a restriction endonuclease recognizes a specific target DNA sequence unless that sequence is methylated by a cognate DNA methyltransferase. Restriction and methyltransferase activities may reside within a single polypeptide (types I and III R-M systems) or separate polypeptides (type II). Restriction enzymes may cut at a site close to (types II and III) or far from (type I) the methylation target sequence. There are also “orphan” methyltransferases, that do not belong to a R-M system. DNA methyltransferases are reviewed extensively in (Murphy et al., 2013), (Casadesús and Low, 2006). Most methyltransferases can use both unmethylated and hemimethylated DNA as substrate, whereas others such as CcrM and Dnmt1 prefer hemimethylated substrates.

Some restriction enzymes possess methyltransferase activity, such as EcoPI15, when SAM is included in the reaction.

Methylation-sensitive nicking endonucleases that specifically recognize unmethylated sites are used in several embodiments. Examples include but are not limited to Nt.AlwI, Nt.BsmAI, Nt.BstNBI. For example, Nt.BstNBI recognizes the sequence GAGTC and is sensitive to (blocked by) adenine methylation (Higgins et al., 2001). HinfI methyltransferase methylates the adenine in GANTC, and can be used to methylate the Nt.BstNBI recognition site. In other embodiments, methylation-sensitive nicking endonucleases that specifically recognize methylated sites can be used (Gutjahr and Xu, 2014).

Methyltransferases are extensively described in (McClelland et al., 1994), (Nelson and McClelland, 1987), (Nelson and McClelland, 1991), (Casadesús and Low, 2006), and (Murphy et al., 2013), which are included herein in their entirety. Sensitivity of restriction enzymes to methylation is described in detail in the New England BioLabs website (https://www.neb.com/tools-and-resources/selection-charts/dam-dcm-and-cpg-methylation), (Nelson and McClelland, 1991), (Nelson and McClelland, 1987), and (McClelland et al., 1994), which are included herein in their entirety.

### Polymerases

Several polymerizing agents can be used in the polymerization reactions described herein. For example, depending on the nucleic acid molecule, a DNA polymerase, an RNA polymerase, or a reverse transcriptase can be used in template-dependent polymerization reactions. DNA polymerases and their properties are described in detail in (Kornberg and Baker, 2005). For DNA templates, many DNA polymerases are available. DNA polymerases with strand-displacing capability are used in several embodiments.

In some embodiments, thermostable polymerases are used, such as Therminator® (New England Biolabs), ThermoSequenase™ (Amersham) or Taquenase™ (ScienTech, St Louis, Mo.).

Useful polymerases can be processive or non-processive. By processive is meant that a DNA polymerase is able to continuously perform incorporation of nucleotides using the same primer, for a substantial length without dissociating from either the extended primer or the template strand or both the extended primer and the template strand. In some embodiments, processive polymerases used herein remain bound to the template during the extension of up to at least 50 nucleotides to about 1.5 kilobases, up to at least about 1 to about 2 kilobases, and in some embodiments at least 5 kb-10 kb, during the polymerization reaction. This is desirable for certain embodiments, for example, where efficient construction of multiple consecutive copies connected to a nucleic acid molecule is performed.

Detailed descriptions of polymerases are found in US 2007/0048748 (Williams et al., 2007), U.S. Pat. No. 6,329,178 (Patel and Loeb, 2001), U.S. Pat. No. 6,602,695 (Patel and Loeb, 2003), U.S. Pat. No. 6,395,524 (Loeb et al., 2002), U.S. Pat. No. 7,981,604 (Quake, 2011), U.S. Pat. No. 7,767,400 (Harris, 2010), U.S. Pat. No. 7,037,687 (Williams et al., 2006), and U.S. Pat. No. 8,486,627 (Ma, 2013) which are incorporated by reference herein.

### Ligases

Adaptors and other nucleic acid constructs can be attached to nucleic acid molecules by using ligation. Several types of ligases are suitable and used in embodiments. Ligases include, but are not limited to, NAD+-dependent ligases including tRNA ligase, Taq DNA ligase, Thermus filiformis DNA ligase, Escherichia coli DNA ligase, Tth DNA ligase, Thermus scotoductus DNA ligase, thermostable ligase, Ampligase thermostable DNA ligase, VanC-type ligase, 9° N DNA Ligase, Tsp DNA ligase, and novel ligases discovered by bioprospecting. Ligases also include, but are not limited to, ATP-dependent ligases including T4 RNA ligase, T4 DNA ligase, T7 DNA ligase, Pfu DNA ligase, DNA ligase 1, DNA ligase III, DNA ligase IV, and novel ligases including wild-type, mutant isoforms, and genetically engineered variants. There are enzymes with ligase activity such as topoisomerases (Schmidt et al., 1994).

### Examples

Methods described herein may employ conventional techniques and descriptions of fields such as organic chemistry, polymer technology, molecular biology, cell biology, and biochemistry, which are within the skill of the art. Such conventional techniques include, but are not limited to, polymerization, hybridization and ligation. Such conventional techniques and descriptions can be found in standard laboratory manuals such as “Genome Analysis: A Laboratory Manual Series (Vols. I-IV)” (Green, 1997), “PCR Primer: A Laboratory Manual” (Dieffenbach and Dveksler, 2003), “Molecular Cloning: A Laboratory Manual” (Green and Sambrook, 2012), and others (Berg, 2006); (Gait, 1984); (Nelson and Cox, 2012), all of which are herein incorporated in their entirety by reference for all purposes.

All referenced publications (e.g., patents, patent applications, journal articles, books) are included herein in their entirety.

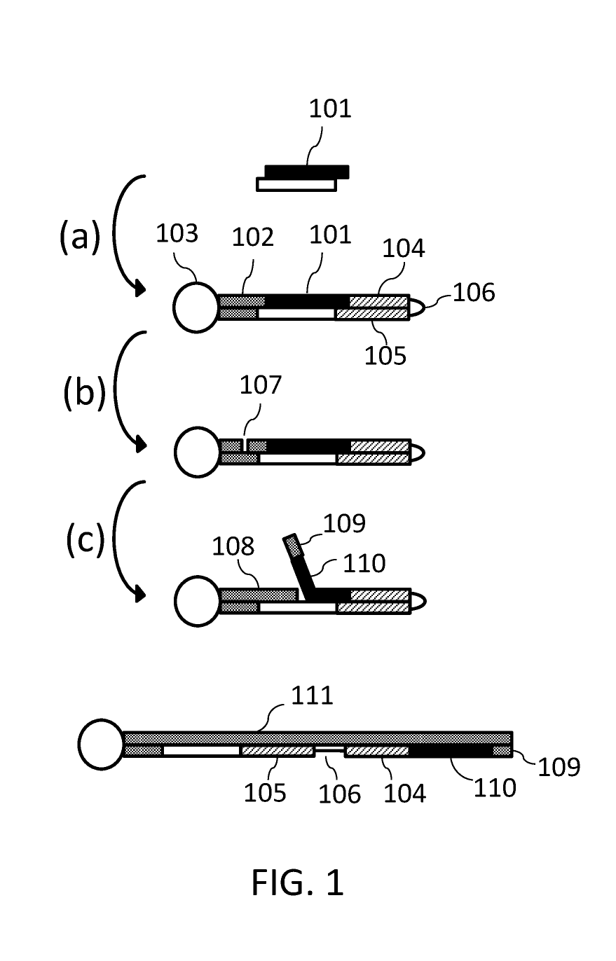

In one embodiment shown in FIG. 1, a nucleic acid molecule 101 is a double-stranded DNA molecule (one strand is drawn white and the other black). 101 comprises overhangs comprising adenine. DNA molecules such as 101 can be generated, for example, by randomly cleaving genomic DNA material, repairing the ends of the resulting DNA fragments, and adding overhangs by incubating with a polymerase such as Taq. All these steps involve methods that are well known to those skilled in the art. In other embodiments, 101 may be blunt-ended.

During step (a), 101 is ligated to an adaptor 102 that is anchored to the surface of a bead 103. In other embodiments, 102 is not anchored. In some other embodiments, 102 may be a hairpin adaptor, with a blunt end or an overhang. In this example, 102 has an overhang comprising thymine and is thus complementary to one of the overhangs in 101. The other end of 101 that is not ligated to 102, is ligated to a hairpin adaptor comprising two at least partially complementary segments 104 and 105, and a loop 106. 105 has an overhang comprising thymine, and is thus complementary to the overhang in 101. In other embodiments, the hairpin adaptor is blunt-ended and ligates to a blunt-ended 101.

The adaptor 102 comprises a cleavable feature. For example, a cleavable feature can be a restriction site for a nicking endonuclease which can create a nick inside or outside the restriction site. In another example, a cleavable feature can be one or more cleavable nucleotides that can lead to the creation of a nick or a gap by using appropriate reagents (e.g. RNases). Cleavable nucleotides and appropriate reagents for cleavage are described in PCT/US2015/027686 which is included herein in its entirety (Tsavachidou, 2015). In this example in FIG. 1, adaptor 102 comprises a nicking endonuclease restriction site. In other embodiments, a cleavable feature may be present in the nucleic acid molecule 101. For example, the nucleic acid molecule may be a construct comprising a genomic fragment pre-attached to an adaptor with a cleavable feature, or a PCR or multiple-displacement amplification product generated using at least one primer comprising a cleavable feature.

During step (b), 101 and its surroundings are subjected to incubation with nicking restriction endonuclease molecules that recognize the specific restriction site within the adaptor 102 and create a nick 107 either within 102 (inside or outside the restriction site) (as shown in FIG. 1), or away from the restriction site and inside 101, or at the end of 102 and the beginning of 101 thus exposing the last 3′ end of 102 (upper strand) and the first 5′ end of 101 (black-colored strand).

In one example, the restriction site within adaptor 102 is methylated, and the nicking restriction endonucleases used in this step recognize only methylated restriction sites, so that any unmethylated restriction sites present in the nucleic acid molecule are not recognized by the endonucleases. In some embodiments, the nick is created within 102, and the sequence between the nick and the beginning of the nucleic acid molecule 101 is specific, for example, to the genomic sample from which the nucleic acid molecule originates, or is an at least partly random sequence unique to the nucleic acid molecule.

During step (c), 101 and its surroundings are exposed to conditions to cause nucleotide incorporation, and to a template-dependent polymerization reaction solution comprising nucleotides and polymerase molecules comprising strand-displacing activity. As shown in FIG. 1, the newly formed segment 108 that starts from nick 107 is displacing the adaptor segment 109 following the nick, and segment 110 of DNA molecule 101. After the polymerization reaction is completed, 108 is fully extended, forming strand 111 which is complementary to 101 (white strand), segment 105 of the hairpin adaptor, loop 106 of the hairpin adaptor, segment 104 of the hairpin adaptor, segment 110 of 101 (black strand) and segment 109 of the adaptor. The product that results from this step has two copies of the DNA molecule 101. Step (c) may optionally include treatment with a reagent (e.g. Taq polymerase or Klenow fragment lacking 3′-5′ exonuclease) that adds an adenine-comprising overhang. Such a treatment may occur concurrently with or following the strand-displacing extension reaction.

The process can be repeated, by ligating another hairpin adaptor (step (a)), nicking (step (b)) and extending with a strand-displacing polymerase (step (c)). The resulting construct will have four copies of 101. Each repetition (cycle) of the process creates a total number of copies of 101 that is double the total number of copies in the previous cycle.

Washing and other treatments may be applied in between described steps as recognized and known by those skilled in the art.

In one related embodiment, the steps in FIG. 1 can be conducted consecutively in each cycle, by washing away reagents used in one step and introducing reagents used in the next step.

In another related embodiment, steps (a) through (c) are carried out in the same reaction, by simultaneously introducing reagents used in all steps, and without washing in between steps. Cycles of copy construction occur within the same reaction. Since washing between steps may not occur in such an embodiment, the copied nucleic acid molecule 101 may not be ligated to an anchored adaptor, or may not be otherwise anchored to a surface.

In other related embodiments, steps (a) through (c) are carried out in the same reaction, by gradually introducing reagents used in one or more steps, and without washing in between steps. Each addition of a reagent or reagents may be followed by inactivation of the added reagent or reagents. Cycles of copy construction occur within the same reaction. Since washing between steps may not occur in such an embodiment, the copied nucleic acid molecule 101 may not be ligated to an anchored adaptor, or may not be otherwise anchored to a surface.

In other related embodiments, steps (a) through (c) are carried out in the same reaction, and may be combined with another step. For example, DNA repair using enzymes such as T4 DNA polymerase or T4 PNK may occur in the same solution, preceding a cycle comprising steps (a) through (c). Such enzymes may be subsequently inactivated.

In other related embodiments, ligations may be blunt-end ligations involving blunt-ended nucleic acid molecules, hairpin adaptors or constructs, or other types of ligations involving overhangs. Those skilled in the art know techniques to create ends suitable for ligation. For example, overhangs may be filled or chewed back to yield blunt ends, in the event that blunt ligation is desired. In another non-limiting example, a polymerase such as Taq is used to create a single-base 3′-end overhang comprising adenine, suitable for TA ligation to an adaptor.

A construct comprising copies of a nucleic acid molecule generated using the method in FIG. 1 can be used for sequencing. For example, such a construct can be detached from surface 103 by using, for example, enzymatic digestion at a specific site within 102. Then, the released construct can be treated appropriately (e.g. incubation with polymerases, A-tailing, etc.) to attach to adaptors appropriate for a nanopore sequencing platform, such as the MinION device (Oxford Nanopore Technologies), and can be subjected to sequencing using such a platform. In other examples, adaptors such as adaptor 102 in FIG. 1 may or may not be anchored to a surface, and may comprise one or more modifications (for example, to allow anchoring to lipid membranes or other surfaces) and/or be linked to one or more enzymes (e.g. helicases) or other molecules. Hairpin adaptors as the one shown in FIG. 1 may also comprise one or more modifications (for example, to allow anchoring to lipid membranes or other surfaces) and/or be linked to one or more enzymes (e.g. helicases) or other molecules. Examples of enzymes that can be linked to adaptors and modifications that are useful for nanopore sequencing are described in PCT/GB2015/050140 and PCT/GB2015/050991 (Heron et al., 2015); (Crawford and White, 2015). The presence of multiple copies within the same construct enables the generation of multiple replicate readings, thereby increasing accuracy, as easily recognized by those skilled in the art.

In another sequencing application, a construct comprising copies of a nucleic acid molecule generated using the method in FIG. 1 can be subjected to circularization, rolling-circle amplification and sequencing using primers specific to sequences within hairpin adaptors within the construct, as easily recognized by those skilled in the art.

In other sequencing applications such as sequencing-by-synthesis or sequencing-by-ligation, a construct comprising copies of a nucleic acid molecule generated using the method in FIG. 1 can be used for sequencing using primers specific to sequences within hairpin adaptors within the construct, as easily recognized by those skilled in the art. The presence of multiple copies within the same construct and their simultaneous sequencing may increase generated optical or electronic or other signal, thereby increasing detection sensitivity, as easily recognized by those skilled in the art.

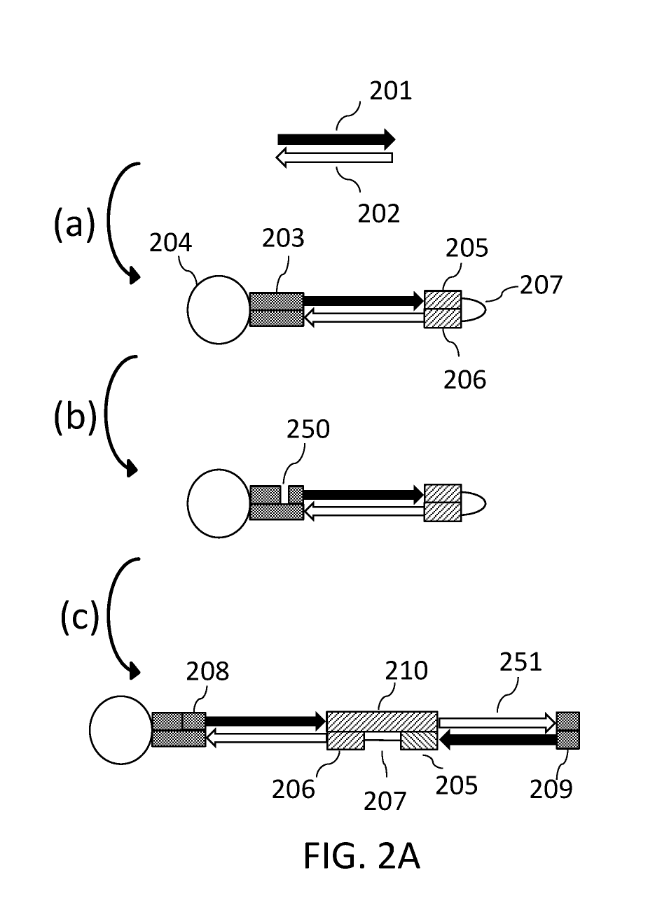

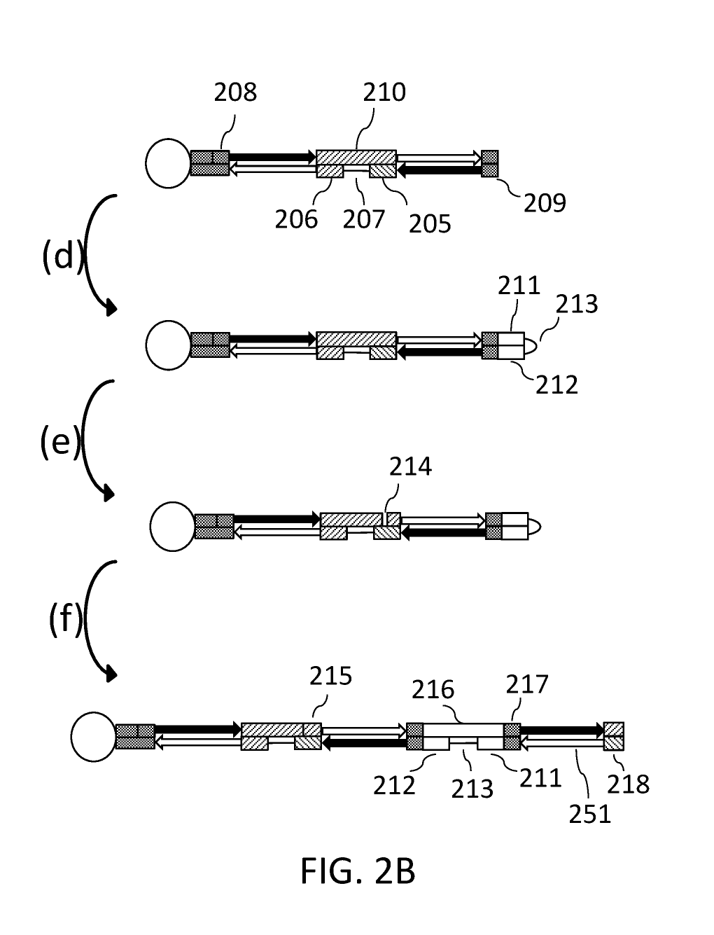

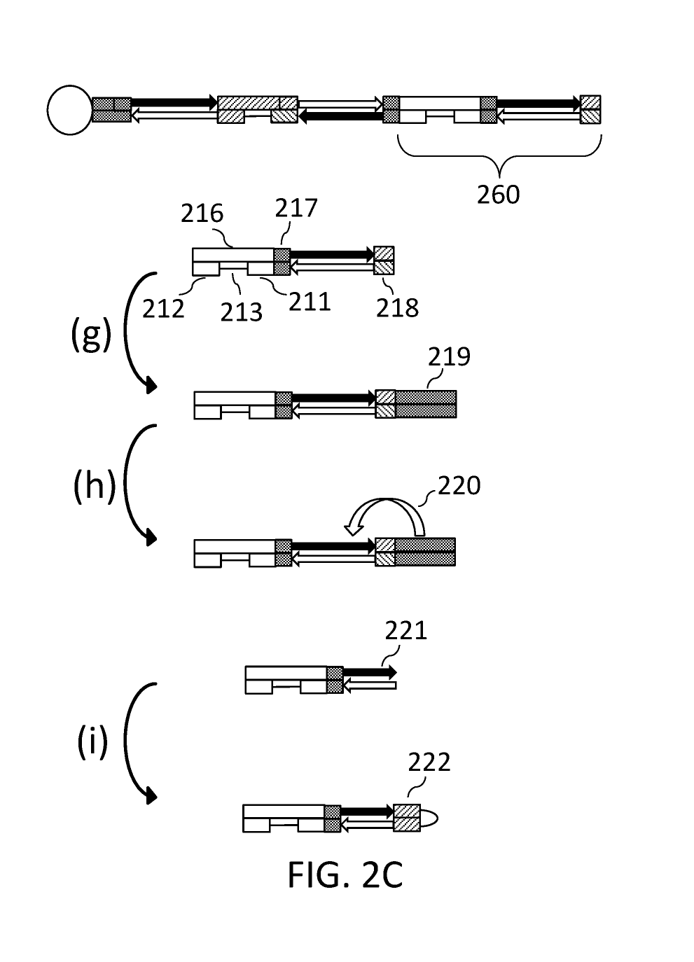

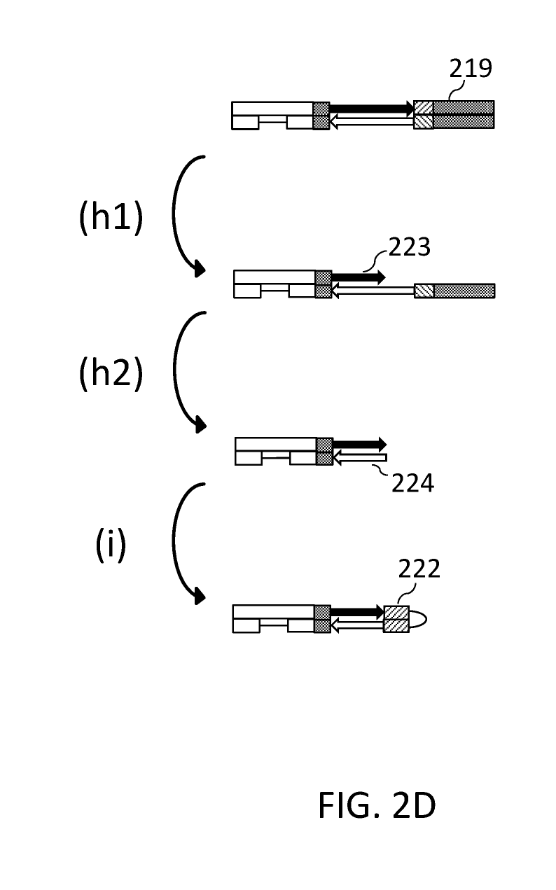

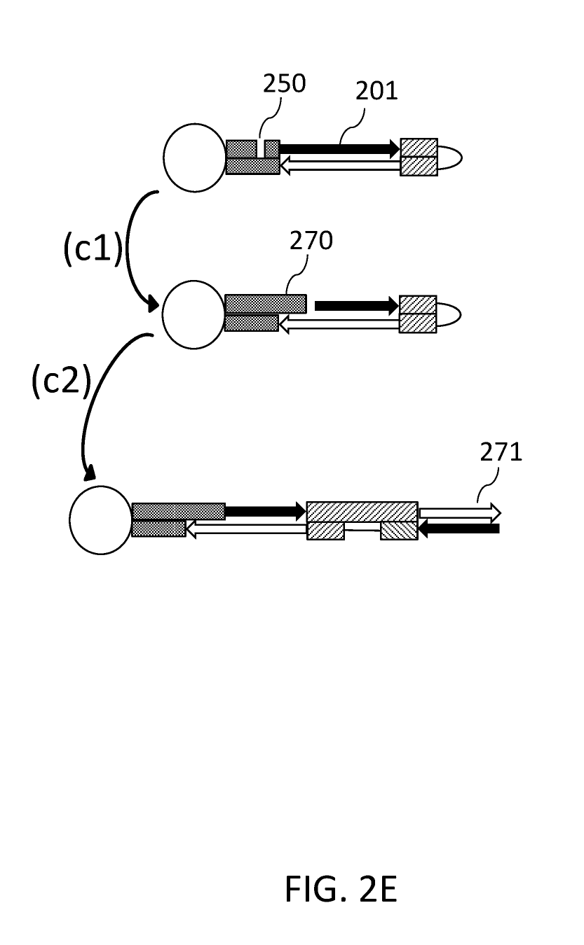

In another embodiment shown in FIG. 2A, a nucleic acid molecule is a blunt-ended double-stranded DNA molecule comprising strand 201 and strand 202. The two strands are represented as arrows demonstrating 5′-to-3′ orientation. DNA molecules such as this can be generated, for example, by randomly cleaving genomic DNA material, and repairing the ends of the resulting DNA fragments.

During step (a), the nucleic acid molecule is ligated to an adaptor 203 that is anchored to the surface of a bead 204. In other embodiments, 203 is not anchored. 203 is blunt in this embodiment. The other end of the nucleic acid molecule that is not ligated to 203, is ligated to a blunt-ended hairpin adaptor comprising two at least partially complementary segments 205 and 206, and a loop 207.

During step (b), the nucleic acid molecule and its surroundings are subjected to incubation with nicking restriction endonuclease molecules that recognize a specific restriction site within the adaptor 203 and create a nick 250 either within 203 (as shown in FIG. 2A), or away from the restriction site and inside strand 201, or at the end of 203 and the beginning of 201 thus exposing the last 3′ end of 203 (upper strand) and the first 5′ end of 201.

During step (c), the nucleic acid molecule and its surroundings are exposed to conditions to cause nucleotide incorporation, and to a template-dependent polymerization reaction solution comprising nucleotides and polymerase molecules comprising strand-displacing activity. The polymerization reaction in step (c): (i) regenerates segment 208 which is the part of the adaptor following the exposed 3′ end at nick 250, (ii) produces a segment complementary to 202, (iii) produces a segment 210 that is complementary to segment 206 of the hairpin adaptor, loop 207 of the hairpin adaptor, and segment 205 of the hairpin adaptor, (iv) produces segment 251 which is complementary to 201, and (v) produces a segment complementary to segment 209, segment 209 having the same sequence (at the 5′-to-3′ direction) with 208, and being inverted in relation to 208. The product that results from this step has two copies of the nucleic acid molecule, one copy being inverted in relation to the other.

In FIG. 2B, the process continues with step (d) which comprises ligating a blunt-ended hairpin adaptor, said hairpin adaptor comprising and least partially complementary segments 211 and 212, and a loop 213.

During step (e), the nucleic acid molecule and its surroundings are subjected to incubation with nicking restriction endonuclease molecules that recognize a specific restriction site within the part of 210 that is complementary to 205 and create a nick 214 within 210.

During step (f), the nucleic acid molecule and its surroundings are exposed to conditions to cause nucleotide incorporation, and to a template-dependent polymerization reaction solution comprising nucleotides and polymerase molecules comprising strand-displacing activity. The polymerization reaction in step (f): (i) regenerates segment 215 which is the part of 210 following the exposed 3′ end at nick 214, (ii) produces a segment complementary to 201 and 209, (iii) produces a segment 216 that is complementary to segment 212 of the hairpin adaptor, loop 213 of the hairpin adaptor, and segment 211 of the hairpin adaptor, (iv) produces segment 217 which is identical to 208, (v) produces a segment complementary to 251, and (v) produces a segment complementary to segment 218, segment 218 having the same sequence (at the 5′-to-3′ direction) with 215, and being inverted in relation to 215. The product that results from this step has three copies of the nucleic acid molecule.

FIG. 2C shows the steps following step (f). For simplicity, clarity and page-fitting purposes, only part 260 is shown in the following steps in FIG. 2C. During step (g), a blunt-ended double-stranded adaptor 219 is ligated to 218 and its complementary segment.

During step (h), the nucleic acid molecule and its surroundings are subjected to incubation with restriction endonuclease molecules that recognize a restriction site within 219. These restriction endonuclease molecules cut outside of their restriction site and inside the nucleic acid molecule copy, as shown by arrow 220. Example of such restriction endonuclease is EcoP15I. Step (h) produces truncated nucleic acid molecule copy 221. The truncated copy may have a blunt end or an end with an overhang, depending on the enzyme that performs the cutting. Those skilled in the art know techniques to create an end suitable for subsequent applications such as ligation to an adaptor. For example, overhangs may be filled or chewed back to yield blunt ends, in the event that blunt ligation is desired. In another non-limiting example, a polymerase such as Taq is used to create a single-base 3′-end overhang comprising adenine, suitable for TA ligation to an adaptor.

In some embodiments, steps (g) and (h) are repeated one or more times using the same or different enzymes, in the event that construction of a shorter copy is desired.

During step (i), the nucleic acid molecule and its surroundings are subjected to a ligation reaction solution and 221 is ligated to hairpin adaptor 222.

In another related embodiment shown in FIG. 2D, truncation of the nucleic acid molecule copy occurs not by using restriction endonucleases, but by performing partial digestion with 3′-to-5′ exonuclease molecules during step (h1), followed by digestion and blunt-end formation during step (h2). During step (h2), the nucleic acid molecule and its surroundings are exposed to a reaction solution comprising 5′-to-3′ exonucleases and/or single-strand-specific endonucleases. Step (h1) generates truncated segment 223, and step (h2) produces truncated segment 224. 223 and 224 are then ligated to hairpin adaptor 222 during step (i).

In another related embodiment, 5′-to-3′ exonucleases such as T7 exonuclease are used instead, during step (h1). During step (h2), 3′-to-5′ exonucleases such as exonuclease I or T, or endonucleases that specifically remove single strands, such as mung bean nuclease, can be used to remove the remaining single-stranded segment of the copy.

In other embodiments, a truncated copy of a nucleic acid molecule may be constructed as shown 770 in FIG. 2E. Instead of performing step (c) as shown in FIG. 2A, a truncated copy 271 is constructed during step (c2) which follows step (c1). Specifically, during step (c1), the nucleic acid molecule and its surroundings are exposed to conditions to cause nucleotide incorporation, and to a template-dependent polymerization reaction solution comprising nucleotides and polymerase molecules. Unlike the polymerase molecules used in step (c) in FIG. 2A, the polymerase molecules in step (c1) exhibit 5′-3′ exonuclease activity. During step (c1), extension starts at nick 250, generating segment 270. The 5′-3′ exonuclease activity of the polymerase molecules leads to digestion of part of the nucleic acid molecule strand 201. In some other embodiments, digestion of part of the nucleic acid molecule can occur by using 5′-3′ exonucleases in step (c1).

During step (c2), the nucleic acid molecule and its surroundings are exposed to conditions to cause nucleotide incorporation, and to a template-dependent polymerization reaction solution comprising nucleotides and polymerase molecules comprising strand-displacing activity. The polymerization reaction in step (c2) produces truncated copy 271 which is inverted in relation to the original nucleic acid molecule.

The length of 271 depends on reagents and conditions used during step (c1). For example, Taq polymerase can be used during step (c1), which performs nucleotide incorporation and at the same time digests the part of strand 201 that it encounters. It is known that Taq polymerase can perform at a speed of >60 nucleotides (nt)/second (sec) at 70° C., 24 nt/sec at 55° C., 1.5 nt/sec at 37° C., and 0.25 nt/sec at 22° C. (Innis et al., 1988). For example, incubation with Taq polymerase at 37° C. for 30 sec may lead to the generation of a truncated copy that is around 1.5*30=45 bases shorter than the original nucleic acid molecule.

Washing and other treatments may be applied in between described steps as recognized and known by those skilled in the art.

In order to generate more copies of a nucleic acid molecule, steps shown in FIG. 2 can be repeated numerous times. After step (i), the process can continue by repeating steps (e) [nicking occurring within the segment 216 that is complementary to the hairpin adaptor ligated during step (d)] and (f), in order to construct an inverted copy of the truncated copy 221. Then, repeating step (d), step (e) [nicking occurring within the segment complementary to the hairpin adaptor ligated during step (i)], step (f), step (g) and step (h) can generate a further truncated copy of the original nucleic acid molecule, that is shorter than 221. A cycle comprising steps (i), (e) [nicking occurring within a segment complementary to the hairpin adaptor ligated during step (d) of the previous cycle], (f), (d), (e) again [nicking occurring within a segment complementary to the hairpin adaptor ligated during step (i) of this cycle], (f) again, (g) and (h) can be repeated several times to generate gradually truncated copies of a nucleic acid molecule.

Washing and other treatments may be applied in between described steps as recognized and known by those skilled in the art.

In other related embodiments, ligations may be TA ligations involving overhangs comprising adenine and thymine, or other types of ligations involving other types of overhangs. Suitable overhangs may be present in nucleic acid molecules, hairpin adaptors or constructs. Those skilled in the art know techniques to create ends suitable for ligation. For example, overhangs may be filled or chewed back to yield blunt ends, in the event that blunt ligation is desired. In another non-limiting example, a polymerase such as Taq is used to create a single-base 3′-end overhang comprising adenine, suitable for TA ligation to an adaptor.

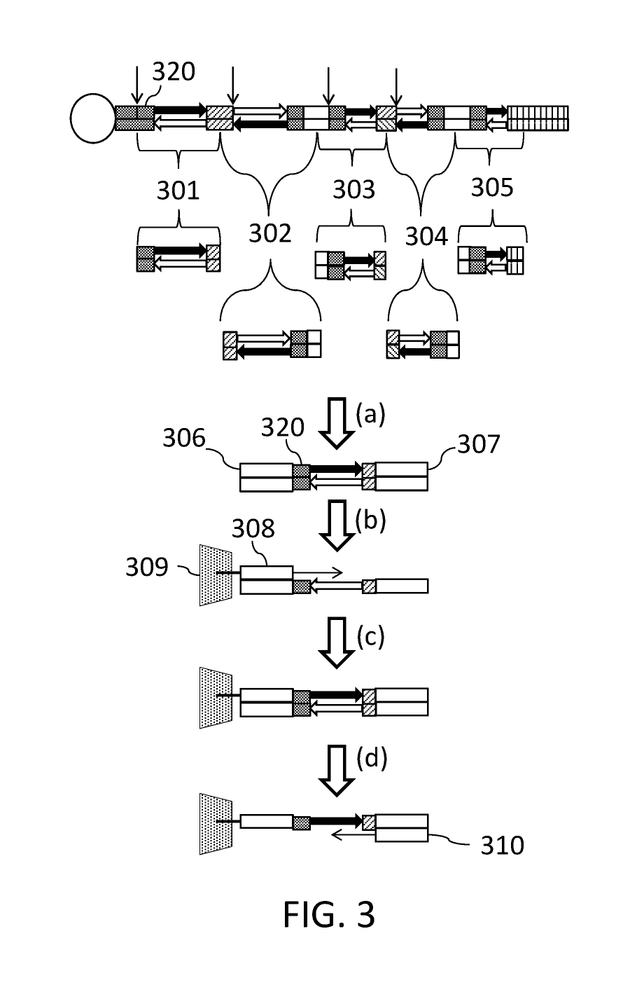

In one embodiment shown in FIG. 3, full-length and truncated copies of a nucleic acid molecule are processed for sequencing. FIG. 3 shows a construct comprising truncated copies of a nucleic acid molecule generated by the process described in the previous figure. The arrows show the positions where nicking occurs during the nicking steps, as described in the previous figure. The segment 320 is copied along with each copy of the nucleic acid molecule. In this embodiment, 320 comprises a specific sequence that serves the role of an “origin identifier” for the nucleic acid molecule and its truncated copies.

After completing the construction of the truncated copies, restriction enzymes can be used to release each of the copies for further processing. In FIG. 3, restriction enzymes recognize and cut restriction sites within adaptor sequences, releasing double-stranded segments 301, 302, 303, 304 and 305. 301 comprises the original nucleic acid molecule, preceded by the origin identifier 320. 302 comprises a full-length copy of the nucleic acid molecule, and a copy of the origin identifier 320. 303 comprises a truncated copy of the nucleic acid molecule, preceded by a copy of the origin identifier 320. The truncation, which is performed during the procedure described in the previous figure, occurs at the side of the nucleic acid molecule not connected to the copy of the origin identifier 320. 304 is the same with 303, shown inverted. 305 comprises a further truncated copy of the nucleic acid molecule, produced by truncating a copy of the already truncated copy in 304. 305 also comprises a copy of the origin identifier 320, which precedes the further truncated copy of the nucleic acid molecule.

Cutting with restriction enzymes may generate blunt ends or overhangs, depending on the type of enzyme used.

During step (a) shown in FIG. 3, the released segments (301, 302, 303, 304 and 305) can be ligated to adaptors. Ligation may occur between blunt ends or overhangs (single-base or more-than-one-base), depending on the ends of the released segments and the ends of the adaptors. Those skilled in the art know techniques to create ends suitable for ligation. For example, overhangs may be filled or chewed back to yield blunt ends, in the event that blunt ligation is desired. In another non-limiting example, a polymerase such as Taq is used to create a single-base 3′-end overhang comprising adenine, suitable for TA ligation to an adaptor. 301 is shown ligated to adaptors 306 and 307. These adaptors may comprise sequences and/or modifications that enable anchoring to surfaces, priming suitable for sequencing, etc. The adaptor-ligated segments may be optionally amplified using PCR with adaptor-specific primers.

During step (b), the construct generated during step (a) is denatured to produce single strands, and then one strand is hybridized to 308, which is an adaptor anchored to a surface 309. 308 can serve as a sequencing primer to initiate sequencing of the strand of 301 serving as the template. The arrow shows the direction of sequencing.

During step (c), sequencing occurs. Full extension of the extending strand can be performed to fully complement the template strand of 301.

During step (d), the newly formed strand is denatured from its template strand, and a new primer 310 is hybridized, to initiate sequencing proceeding at the direction opposite from that of step (c). The arrow shows the direction of sequencing.

Washing and other treatments may be applied in between described steps as recognized and known by those skilled in the art.

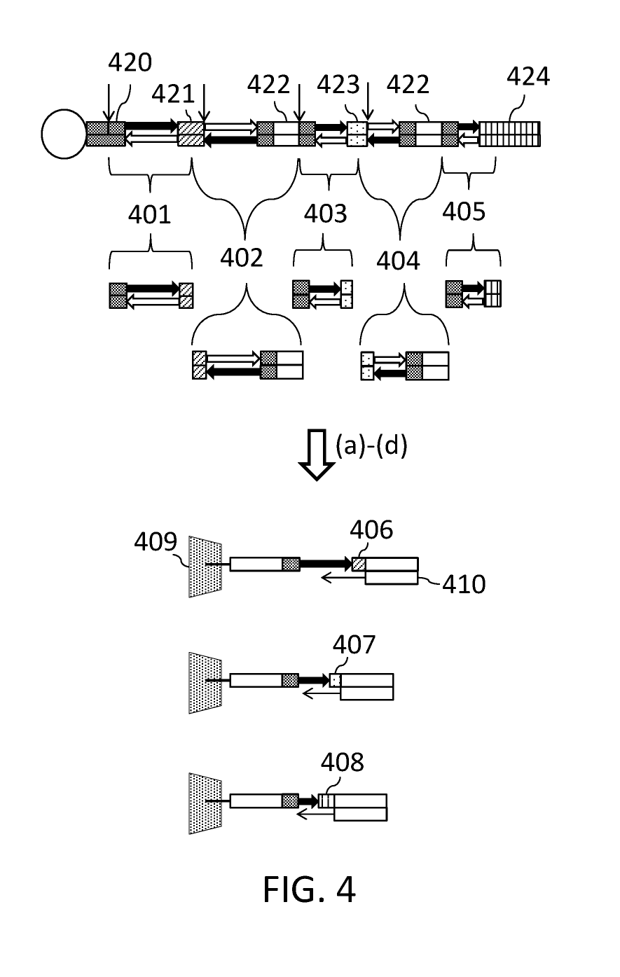

In FIG. 4, a construct is shown which is similar to the one in FIG. 3, comprising truncated copies of a nucleic acid molecule generated by the process described in FIG. 2. The arrows show the positions where nicking occurs during the nicking steps, said nicking steps occurring as described in FIG. 2. The segment 420 is copied along with each copy of the nucleic acid molecule. In this embodiment, 420 comprises a specific sequence that serves the role of an origin identifier for the nucleic acid molecule and its truncated copies. Additionally, adaptors 421, 423 and 424 comprise sequences termed “copy identifiers”, each of which is specific to a specific truncated copy.

After completing the construction of the truncated copies, restriction enzymes can be used to release each of the copies for further processing. In FIG. 4, restriction enzymes recognize and cut restriction sites within adaptor sequences, releasing double-stranded segments 401, 402, 403, 404 and 405.

In this embodiment, segments 401 and 402 comprise identical copies of the nucleic acid molecule, a copy of the origin identifier 420, and a part of the hairpin adaptor 421 which is a copy identifier specific to the full-length copy of the nucleic acid molecule. Similarly, segments 403 and 404 comprise the same type of truncated nucleic acid molecule copy, a copy of the origin identifier 420, and a part of the hairpin adaptor 423 which is a copy identifier specific to the specific truncated copy of the nucleic acid molecule. Segment 405 comprises a further truncated copy of the nucleic acid molecule, a copy of the origin identifier 420, and a part of the adaptor 424 which is a copy identifier specific to this further truncated copy of the nucleic acid molecule.

Similarly to the segments in FIG. 3, the segments in FIG. 4 are subjected to steps (a) through (d) as described in FIG. 3. Three single-stranded copies are shown in FIG. 4, each originating from segments 401, 403 and 405 respectively. These single-stranded copies are attached to a surface 409, and primer 410 is hybridized to each single-stranded copy to allow sequencing toward the direction demonstrated by the arrows (step (d), as described in FIG. 3). Sequencing during step (d) yields the sequences of the fragments 406, 407 and 408, and the sequences of the 3′ end of the nucleic acid molecule copies that previously participated in truncation steps. 406, 407 and 408 are copy identifiers which originated from the adaptors 421, 423 and 424 respectively. Sequencing of 406, 407 and 408 is particularly useful during short-read sequencing, because the sequences of these fragments can identify the order with which the sequenced 3′ ends of the nucleic acid molecule copies can be arranged in the proper order to reconstruct the sequence of the original full-length nucleic acid molecule. Sequence arrangements can be performed using bioinformatics methods well-known to those skilled in the art.

In the event that copies from multiple nucleic acid molecules are sequenced, the origin identifier 420 which is present in each copy originating from the same nucleic acid molecule enables arranging together only the sequences from copies originating from the same nucleic acid molecule. During the sequencing step (c) described in detail in FIG. 3, sequencing of 420 is enabled.

Identification of specific sequences, sequence arrangements and other related analyses can be performed using bioinformatics methods well-known to those skilled in the art.

Washing and other treatments may be applied in between described steps as recognized and known by those skilled in the art.

In some embodiments, fragments 301, 302, 303, 304 and 305 in FIG. 3 and fragments 401, 402, 403, 404 and 405 in FIG. 4 are generated by using multiplex PCR comprising appropriate primers as easily recognized by those skilled in the art.

Hairpin adaptors may comprise one or more restriction sites. Such restriction sites enable recognition and cutting by restriction endonucleases or nicking endonucleases. Restriction enzymes and nicking endonucleases may cut inside or outside of their restriction site. Restriction enzymes may create blunt or sticky ends. In the event that more than one restriction sites are present within the same hairpin adaptor, they may be separate or overlapping. Restriction sites or parts thereof may be located within the loop of the hairpin adaptor, or within at least partially complementary segments of the hairpin adaptor, or within segments of the hairpin adaptors comprising at least one mismatch, said mismatch being single-base or comprising more than one base. Hairpin adaptors may comprise at least part of a primer sequence and/or adaptor sequence that can be used during sequencing (for example, sequence that enables anchoring to a surface, or sequence that enables primer hybridization). Hairpin adaptors may, for example, have blunt ends or a 5′ end overhang or a 3′ end overhang or at least partially non-complementary 5′ and 3′ ends.

Hairpin adaptors used during the same procedure may comprise the same or different sequences, and/or the same or different restriction sites.

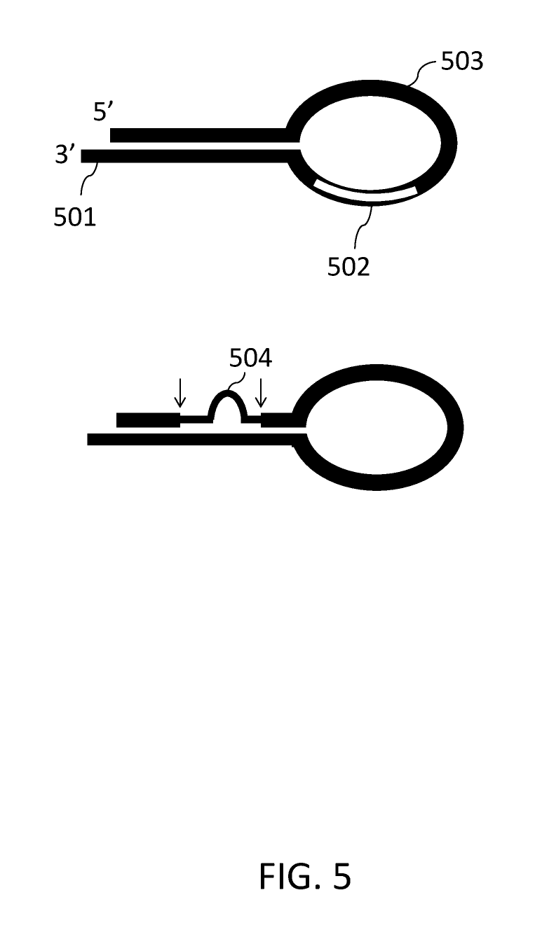

A non-limiting example of a hairpin adaptor is shown in FIG. 5. This hairpin adaptor has a 3′ end overhang 501, and a loop 503. Within the loop, there is a single-stranded part 502 of a restriction site which can be a nicking enzyme recognition site, or a restriction endonuclease site. Since the loop is a non-complementary region, 502 cannot be recognized by its corresponding restriction enzyme when the hairpin adaptor is folded. In the event that a strand complementary to the hairpin is constructed, 502 becomes a double-stranded segment and can be recognized by its corresponding restriction enzyme. Another non-limiting example of a hairpin adaptor is shown in FIG. 5, wherein a mismatch 504 is positioned within the, otherwise, self-complementary part of the hairpin adaptor. 504 is positioned within a site marked with a thinner line, whose borders are pointed by arrows. This site represents (i.e., is the single-stranded part of) a restriction site. Because of the mismatch, the site cannot be recognized by its corresponding restriction enzyme while the hairpin adaptor is folded. In the event that a strand complementary to the hairpin is constructed, the thinner-lined segment becomes a double-stranded segment and can be recognized by its corresponding restriction enzyme, whereas its mismatched counterpart remains unable to be recognized by the restriction enzyme. Instead of a mismatch, a modification can be used (for example, one or more methylated nucleotides) to inhibit recognition by a restriction enzyme.

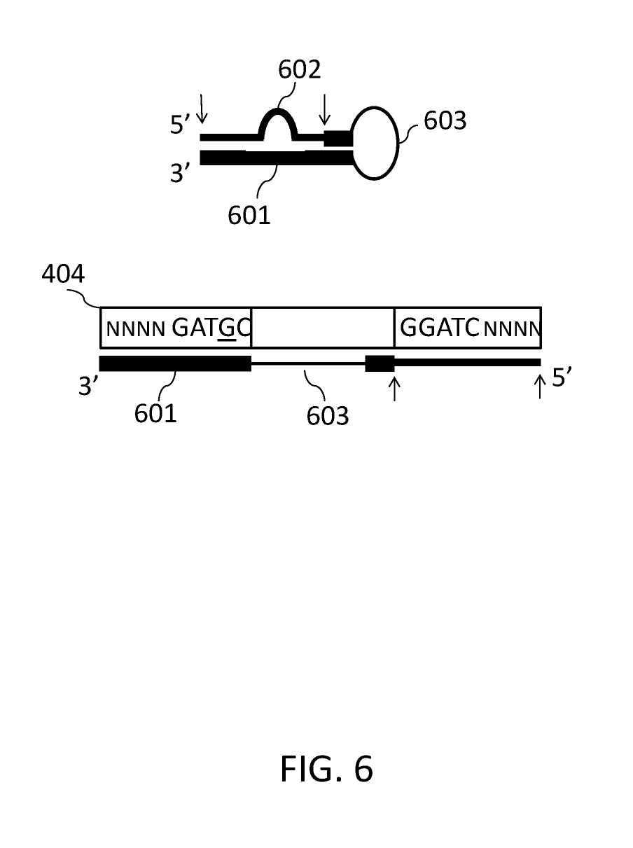

Another non-limiting example of a hairpin adaptor comprising a mismatch is shown in FIG. 6. The hairpin adaptor shown in FIG. 6 has a loop 603, a segment 601 and another segment complementary to 601 with the exception of a single-base mismatch 602. The thin-lined segment whose borders are pointed by arrows represents two overlapping restriction sites. As in the example in FIG. 5, the mismatch prevents recognition by restriction enzymes while the hairpin adaptor is in folded conformation. Instead of a mismatch, a modification can be used (for example, one or more methylated nucleotides) to inhibit recognition by a restriction enzyme.

In the event that a strand 404 complementary to the hairpin is constructed, the thinner-lined segment becomes a double-stranded segment leading to a fully formed restriction site that can be recognized by its corresponding restriction enzymes, whereas its mismatched counterpart 601 remains unable to be recognized by the restriction enzymes. In the non-limiting example shown in FIG. 6, the overlapping restriction sites are GGATCNNNN recognized by the nicking endonuclease Nt.AlwI, and GATC recognized by DpnII. The mismatch 602 is the underlined G shown within the segment of 404 that is complementary to 601. 602 renders this segment non-recognizable by the enzymes, thus preventing any unwanted nicking or cutting.

For example, in an embodiment similar to the one shown in FIGS. 2A through 2C, the hairpin adaptors ligated during steps (a), (d) and (i) have a structure similar to the one described in FIG. 6. In addition, after steps (b) and (e) and before steps (c) and (f) respectively, the nucleic acid molecule and its surroundings are exposed to a reaction solution comprising dam methyltransferases. Dam methyltransferases recognize the GATC sequence and methylate the adenine within this sequence. The mismatches within the GATC site of the hairpin adaptors (as described in FIG. 6) prevent unwanted recognition and methylation by dam methyltransferases while the hairpin adaptors are in folded conformation. Methylation-sensitive enzymes such as Nt.AlwI can introduce nicks within said hairpin adaptors when they are rendered double-stranded and are no longer in folded conformation, only in the one (desired) side of the double-stranded hairpin adaptor. Additionally, such methylation-sensitive enzymes do not recognize methylated sites within double-stranded hairpin adaptors. Specifically, in the above-described embodiment comprising methylation steps, DNA methylation after step (b) does not methylate the hairpin adaptor comprising a mismatch within GATC, said hairpin adaptor being in folded conformation and being ligated to the nucleic acid molecule during step (a). So, during step (e), a nick 214 forms within said hairpin adaptor. Moreover, methylation after step (b) renders adaptor 203 methylated and prevents undesirable nicking by a methylase-sensitive enzyme (such as Nt.AlwI) during step (e). Using methyltransferases prevents undesirable cutting not only within adaptors but also within copies of nucleic acid molecules. In another embodiment similar to the one described above, an additional step before step (g) occurs, said step comprising exposing the nucleic acid molecule and its surroundings to a reaction solution comprising EcoP15I and SAM (S-adenosyl methionine). During this additional step, the nucleic acid molecule and its surroundings are methylated at EcoP15I sites, to prevent undesirable recognition and cutting by EcoP15I during step (h).

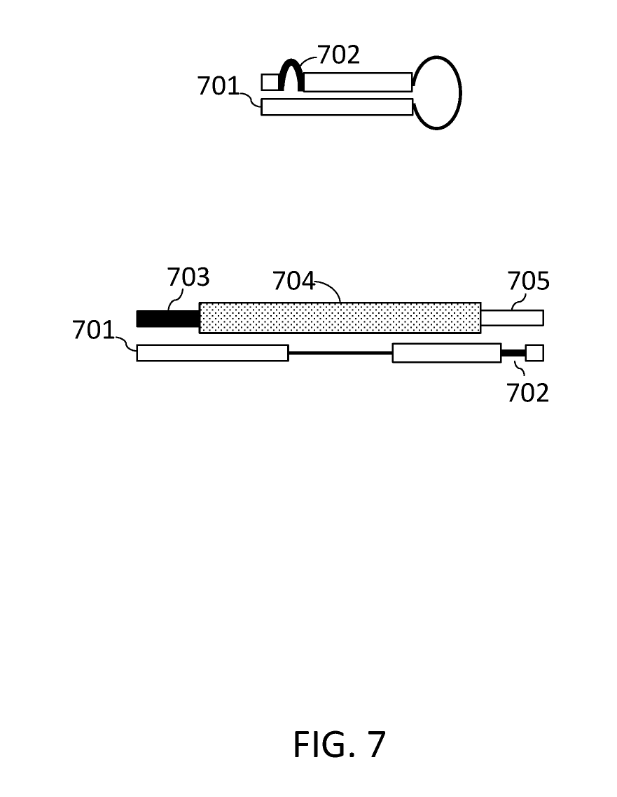

Another non-limiting example of a hairpin adaptor is shown in FIG. 7. Similarly to the hairpin adaptor in FIG. 6, this hairpin adaptor has a segment 701 and another segment complementary to 701 with the exception of a mismatch 702. In the event that a strand complementary to the hairpin is constructed, segment 703 is complementary to 701 and corresponds to a restriction site which becomes recognizable by its corresponding restriction enzyme. Similarly, segment 705 is complementary to the hairpin segment comprising 702 and corresponds to a restriction site, which also becomes recognizable by its corresponding restriction enzyme, which restriction enzyme is different from the restriction enzyme recognizing 703. Segment 704 may comprise primer and/or adaptor sequences useful for sequencing.

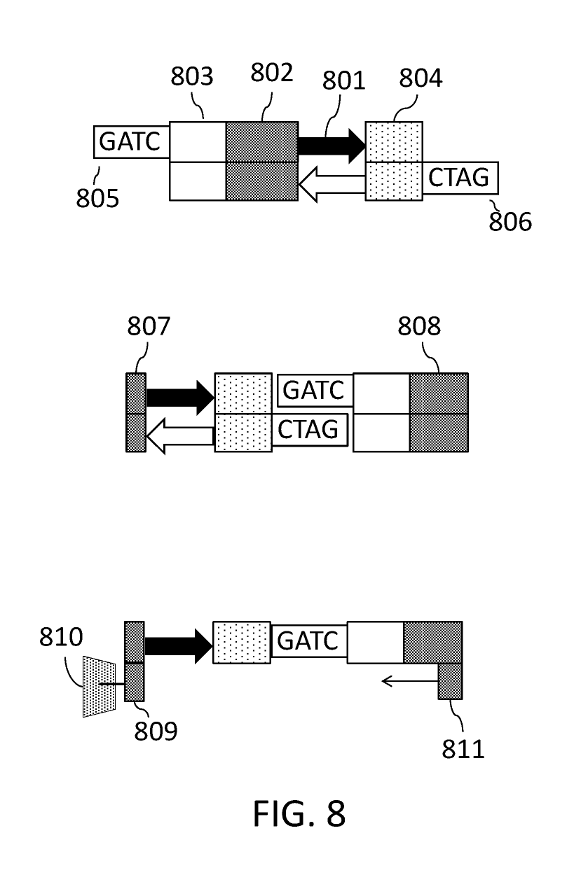

As described previously herein, copies of a nucleic acid molecule can be released for further processing by using restriction enzymes. FIG. 8 shows an example of a construct comprising a copy 801 being attached to an origin identifier 802, and a copy identifier 804. 804 is part of an adaptor which is previously subjected to restriction enzyme cutting by DpnII, leading to the formation of the overhang 806 (CTAG). 802 is also attached to 803 which is part of an adaptor which is previously subjected to restriction enzyme cutting by DpnII, leading to the formation of the overhang 805 (GATC).

In addition to serving as an origin identifier, 802 also comprises a restriction site, an adaptor anchoring site and a site for primer hybridization. Since 805 and 806 are complementary, an appropriate ligation reaction that can be performed by anyone skilled in the art can lead to circularization of the construct. Subsequently, restriction enzymes recognizing the restriction site within 802 can linearize the circular product, giving rise to a linear segment flanked by segments 807 and 808. 807 and 808 are parts of 802. The linear product can be denatured and processed for sequencing. Specifically, 807 comprises an adaptor anchoring sequence that can hybridize to adaptor 809 which is linked to a surface 810, thus anchoring the denatured linear product to the surface. Then, primer 811 hybridizes to a complementary site within 808, thus initiating sequencing towards the direction shown by the arrow. 808 also comprises the origin identifier sequence within 802, so that sequencing initiated by 811 may cover the origin identifier, the copy identifier and the 3′ end of 801. Unlike the sequencing methods described previously herein, the method in FIG. 8 enables sequencing of the origin identifier, the copy identifier and the 3′ end of the nucleic acid molecule copy in a single sequencing read, and not in two separate paired reads.

Washing and other treatments may be applied in between described steps as recognized and known by those skilled in the art.

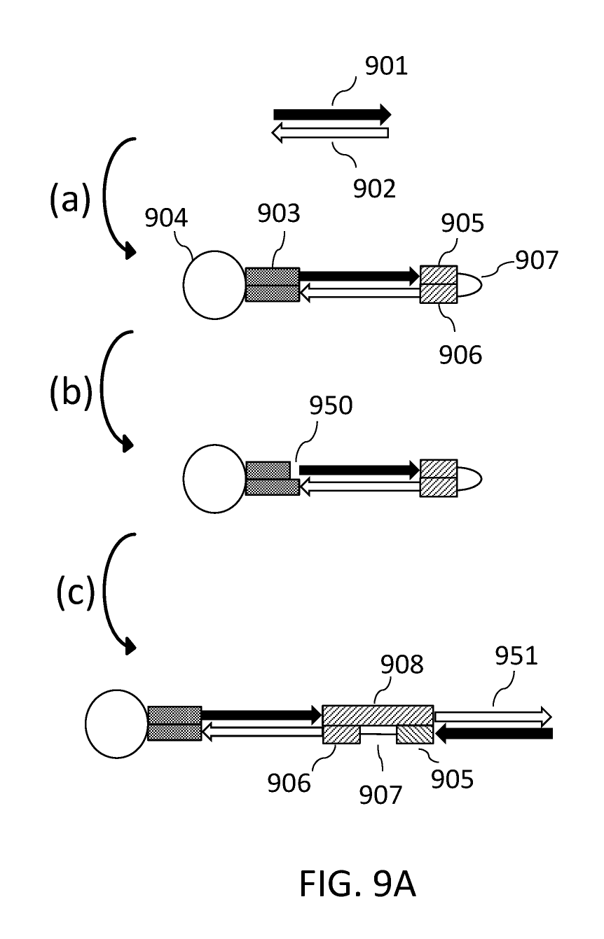

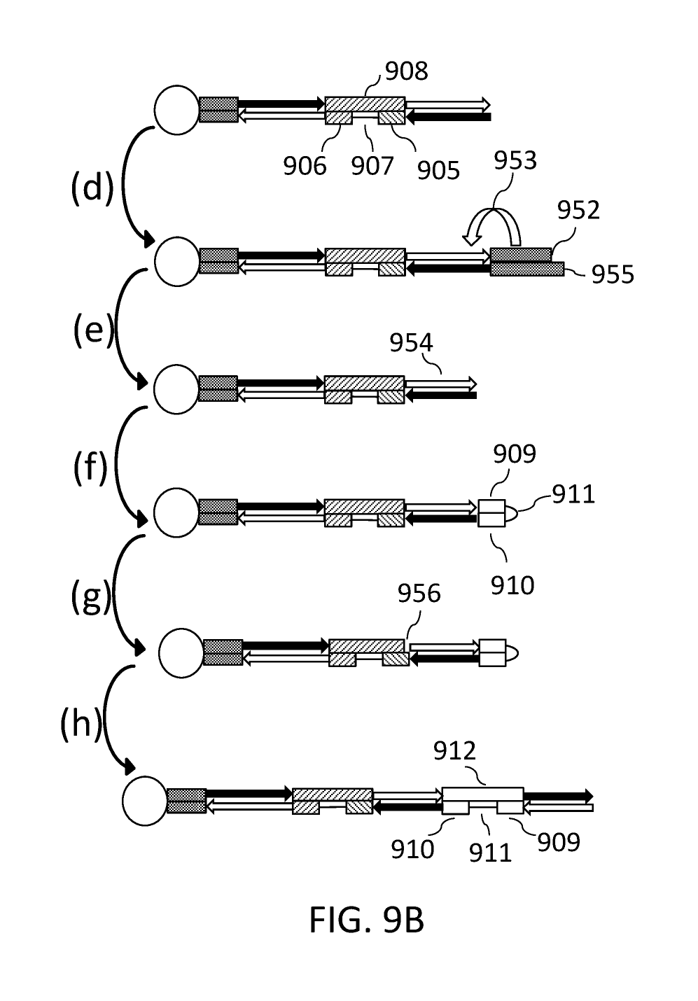

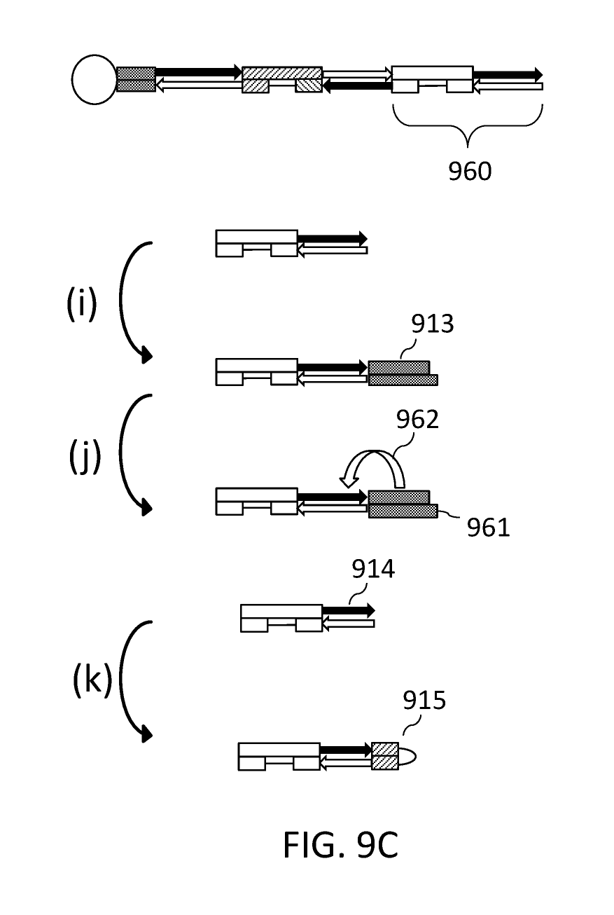

In another embodiment shown in FIG. 9A, a nucleic acid molecule is a blunt-ended double-stranded DNA molecule comprising strand 901 and strand 902. The two strands are represented as arrows demonstrating 5′-to-3′ orientation. DNA molecules such as this can be generated, for example, by randomly cleaving genomic DNA material, and repairing the ends of the resulting DNA fragments.

During step (a), the nucleic acid molecule is ligated to an adaptor 903 that is anchored to the surface of a bead 904. In other embodiments, 903 is not anchored. 903 is blunt in this embodiment. The other end of the nucleic acid molecule that is not ligated to 903, is ligated to a blunt-ended hairpin adaptor comprising two at least partially complementary segments 905 and 906, and a loop 907.

During step (b), the nucleic acid molecule and its surroundings are subjected to incubation with nicking restriction endonuclease molecules that recognize a specific restriction site within the adaptor 903 and create a nick 950 exposing the last 3′ end of 903 (upper strand) and the first 5′ end of the nucleic acid molecule (strand 901). In other embodiments, the nick is within 903, or within the nucleic acid molecule.

During step (c), the nucleic acid molecule and its surroundings are exposed to conditions to cause nucleotide incorporation, and to a template-dependent polymerization reaction solution comprising nucleotides and polymerase molecules comprising strand-displacing activity. The polymerization reaction in step (c): (i) produces a segment complementary to 902, (ii) produces a segment 908 that is complementary to segment 906 of the hairpin adaptor, loop 907 of the hairpin adaptor, and segment 905 of the hairpin adaptor, and (iii) produces segment 951 which is complementary to 901. The product that results from this step has two copies of the nucleic acid molecule, one copy being inverted in relation to the other.

In FIG. 9B, the process continues with step (d) which comprises ligating an adaptor 952. During step (e), the nucleic acid molecule and its surroundings are subjected to incubation with restriction endonuclease molecules that recognize a restriction site within 952. These restriction endonuclease molecules cut outside of their restriction site and inside the nucleic acid molecule copy, as shown by arrow 953. Example of such restriction endonuclease is EcoP15I. Step (e) produces truncated nucleic acid molecule copy 954. The truncated copy may have a blunt end or an end with an overhang, depending on the enzyme that performs the cutting. Those skilled in the art know techniques to create an end suitable for subsequent applications such as ligation to an adaptor. For example, overhangs may be filled or chewed back to yield blunt ends, in the event that blunt ligation is desired. In another non-limiting example, a polymerase such as Taq is used to create a single-base 3′-end overhang comprising adenine, suitable for TA ligation to an adaptor.

The adaptor 952 may have an overhang or recessive end or modification at the 3′ end 955, which may prevent ligation of hairpin or other adaptors during future steps, in the event that enzymatic cleavage during step (e) is incomplete.

In some embodiments, steps (d) and (e) are repeated one or more times using the same or different enzymes, in the event that construction of a shorter copy is desired.

During step (f), the truncated copy 954 is ligated to a hairpin adaptor comprising two at least partially complementary segments 909 and 910, and a loop 911.

During step (g), the nucleic acid molecule and its surroundings are subjected to incubation with nicking restriction endonuclease molecules that recognize a specific restriction site within the part of 908 that is complementary to 905 and create a nick 956 between the end of 908 and the beginning of 954. In other embodiments, the nick may be within 908, or within 954. In other embodiments, the restriction site may be within a different part of 908.

During step (h), the nucleic acid molecule and its surroundings are exposed to conditions to cause nucleotide incorporation, and to a template-dependent polymerization reaction solution comprising nucleotides and polymerase molecules comprising strand-displacing activity. The polymerization reaction in step (h) produces a segment 912 that is complementary to segment 910 of the hairpin adaptor, loop 911 of the hairpin adaptor, and segment 909 of the hairpin adaptor. It also produces a copy of the truncated copy 954, which is inverted in relation to 954. The overall product that results from this step has three copies of the nucleic acid molecule (the original nucleic acid molecule, and two truncated copies).

FIG. 9C shows the steps following step (h). For simplicity, clarity and page-fitting purposes, only part 960 is shown in the following steps in FIG. 9C. During step (i), a double-stranded adaptor 913 is ligated to the copy generated during step (h).

During step (j), the nucleic acid molecule and its surroundings are subjected to incubation with restriction endonuclease molecules that recognize a restriction site within 913. These restriction endonuclease molecules cut outside of their restriction site and inside the nucleic acid molecule copy, as shown by arrow 962. Example of such restriction endonuclease is EcoP15I. Step (j) produces truncated nucleic acid molecule copy 914. The truncated copy may have a blunt end or an end with an overhang, depending on the enzyme that performs the cutting. Those skilled in the art know techniques to create an end suitable for subsequent applications such as ligation to an adaptor. For example, overhangs may be filled or chewed back to yield blunt ends, in the event that blunt ligation is desired. In another non-limiting example, a polymerase such as Taq is used to create a single-base 3′-end overhang comprising adenine, suitable for TA ligation to an adaptor.

The adaptor 913 may have an overhang or recessive end or modification at the 3′ end 961, which may prevent ligation of hairpin or other adaptors during future steps, in the event that enzymatic cleavage during step (j) is incomplete.

In some embodiments, steps (i) and (j) are repeated one or more times using the same or different enzymes, in the event that construction of a shorter copy is desired.

During step (k), the nucleic acid molecule and its surroundings are subjected to a ligation reaction solution and 914 is ligated to hairpin adaptor 915.

Washing and other treatments may be applied in between described steps as recognized and known by those skilled in the art.

In some embodiments, methylation steps may follow steps (b), (c), (g) and (h), as described in a previous paragraph herein for an embodiment similar to the one described in FIGS. 2A through 2C.

After step (k), the process can continue by repeating steps (b) through (f) one or more times, to generate progressively truncated copies of a nucleic acid molecule.

Washing and other treatments may be applied in between described steps as recognized and known by those skilled in the art.

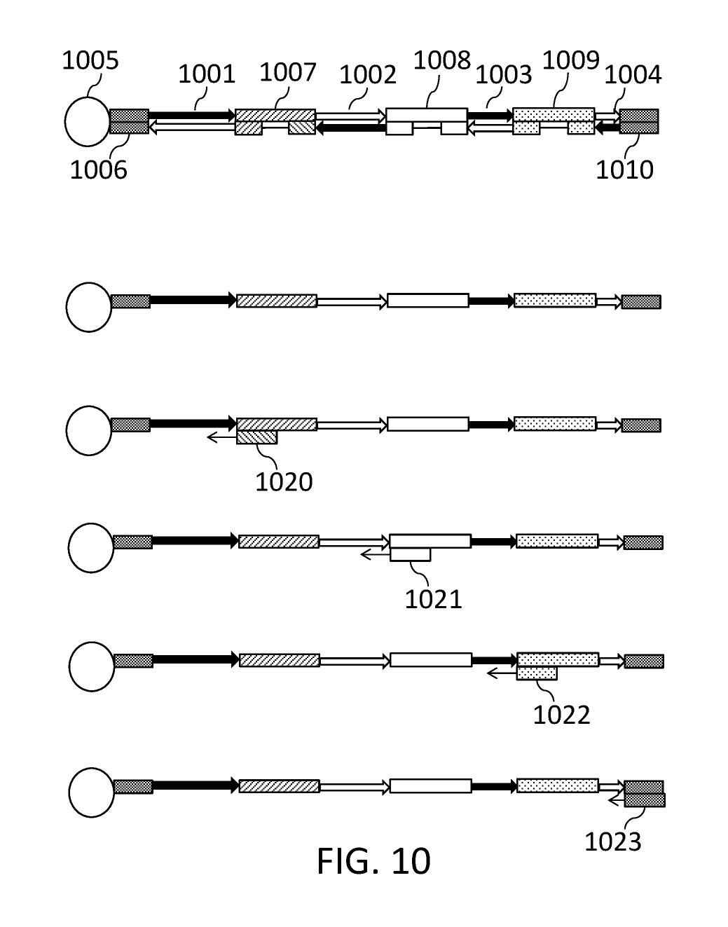

In one embodiment shown in FIG. 10, full-length and truncated copies of a nucleic acid molecule are processed for sequencing. FIG. 10 shows a construct comprising truncated copies of a nucleic acid molecule 1001 generated by the process described in the previous figure. Copy 1002 is shorter than 1001, copy 1003 is shorter than 1002, and copy 1004 is shorter than 1003. 1001 is ligated to adaptor 1006 which is anchored to a solid support 1005. Hairpin adaptors 1007, 1008 and 1009, and adaptor 1010 have distinct sequences, different from one another.

First, denaturation conditions are applied to create a single-stranded construct, which is exposed to sequencing primers 1020. Primer 1020 anneals to a sequence within 1007. Sequencing proceeds to the direction of the arrow.

After sequencing using primer 1020 is completed, annealing of another primer, 1021, may occur. Primer 1021 anneals to a sequence within 1008, initiating sequencing towards the direction of the arrow. After sequencing using primer 1021 is completed, annealing of another primer, 1022, 1105 may occur. Primer 1022 anneals to a sequence within 1009, initiating sequencing towards the direction of the arrow. After sequencing using primer 1022 is completed, annealing of another primer, 1023, may occur. Primer 1023 anneals to a sequence within 1010, initiating sequencing towards the direction of the arrow.

It becomes clear to those skilled in the art that sequencing short parts of the progressively truncated copies 1002, 1003 and 1004 can reveal a part of the sequence of 1001 that is significantly longer than the sequence that can be retrieved by sequencing 1001 alone. For example, in one embodiment, 1003 is constructed by truncating 1001 using EcoP15I. EcoP15I removes 27 bases, so that 1003 is 27 bases shorter than 1001. Also, in this example, we use a sequencing method that accomplishes 27-base reads, so that sequencing initiated by primer 1020 retrieves 27 bases of 1001, and sequencing initiated by primer 1022 retrieves 27 bases of 1003. This way, we retrieve a part of the sequence of 1001 comprising 2*27=54 bases, instead of the only 27 bases that we would get by sequencing 1001 alone.

In some embodiments, the construct shown in FIG. 10 is amplified prior to sequencing, by using bridge amplification for example, to generate colonies.

In other embodiments, the construct shown in FIG. 10 is not anchored to a surface, but is instead circularized, subjected to rolling-circle amplification and subsequently sequenced.

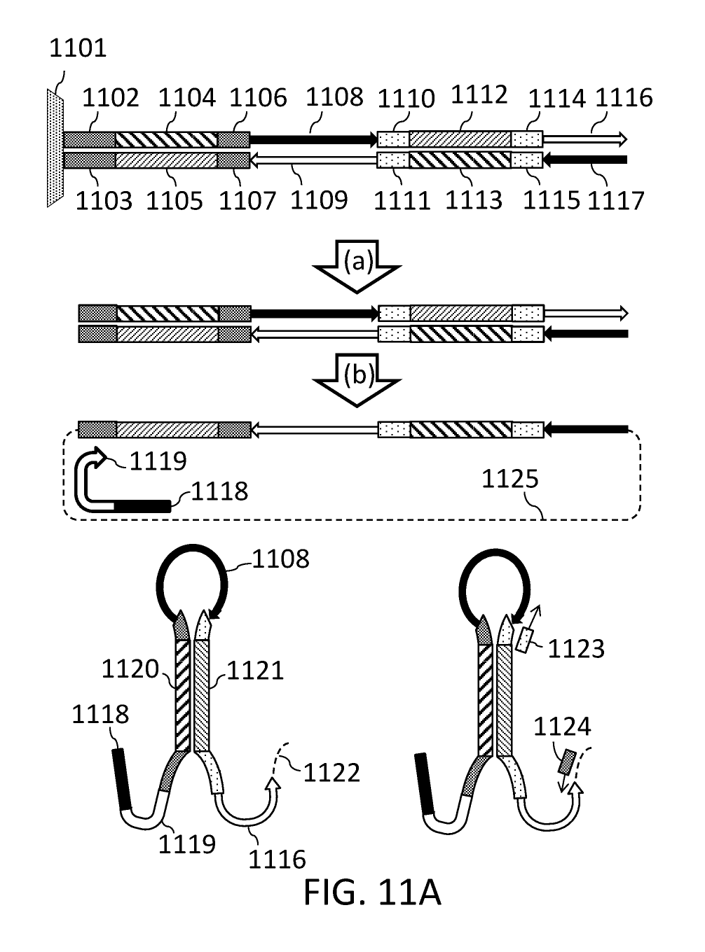

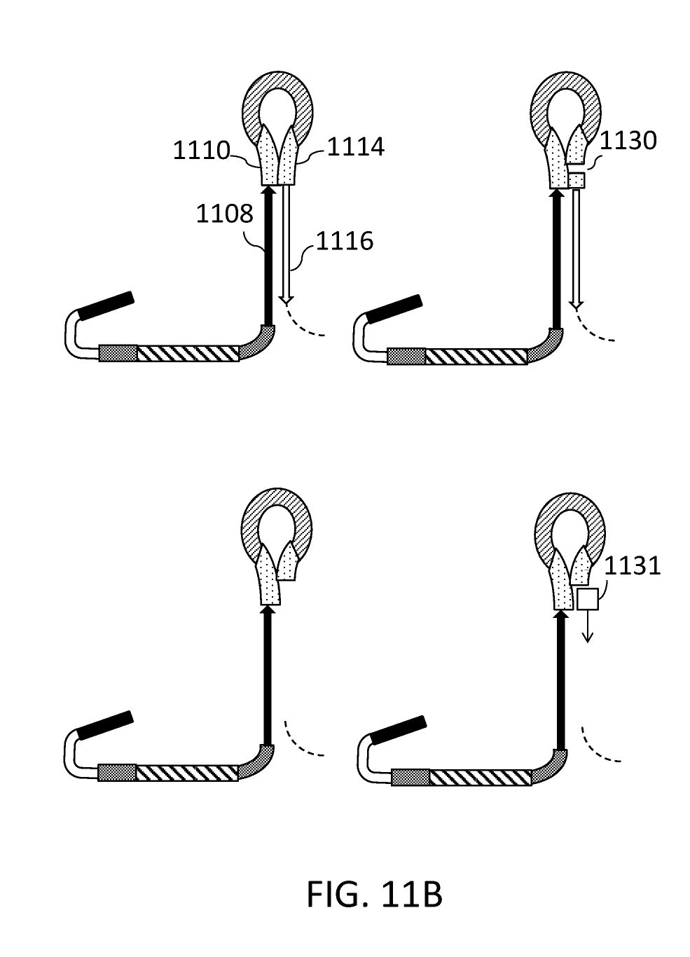

In some embodiments, consecutively constructed and progressively truncated copies can be amplified using rolling-circle amplification (RCA) and sequenced. In one embodiment shown in FIG. 11A, a double-stranded DNA construct comprising strands 1116 and 1117 is a truncated copy of a nucleic acid molecule comprising strands 1108 and 1109. As described in previous figures, the truncated copy is inverted in relation to the original nucleic acid molecule, so that 1116 is complementary to 1108, and 1117 is complementary to 1109. The nucleic acid molecule is attached to an adaptor immobilized to a surface 1101; the adaptor comprises segments 1102, 1104 and 1106, and their complementary segments 1103, 1105 and 1107 respectively. The nucleic acid molecule and its truncated copy are attached to a hairpin adaptor comprising segments 1110, 1112 and 1114, and to the hairpin adaptor's complementary strand comprising segments 1111, 1113 and 1115, where 1111 is complementary to 1110, 1113 is complementary to 1112, and 1115 is complementary to 1114. 1112 is the hairpin adaptor's loop. The adaptor and the hairpin adaptor can be made so that 1112 is complementary to 1104.

During step (a), the adaptor is released from surface 1101. Methods of release depend on the nature of the connection between the adaptor and the surface, and/or the design of the adaptor, and are well-known to those skilled in the art. For example, restriction enzymes recognizing a site within the adaptor can be used to cleave said site and release the adaptor.

During step (b), the released product can be denatured and circularized. Circularization may precede or follow denaturation. Circularization may involve direct ligation of the adaptor and the truncated copy, or ligation to the ends of another construct (e.g., vector). In FIG. 11A, circularization is accomplished by ligating to vector 1125 (dashed line). Subsequently, primer 1118 anneals to 1125 and initiates RCA towards the direction shown by the extension 1119 of the primer. RCA protocols are well known to those skilled in the art. RCA yields a long single-stranded product that can be used for sequencing, using unchained sequencing by combinatorial probe anchor ligation (cPAL), for example (Drmanac et al., 2010).

A potential problem arising from the single-stranded nature of the RCA-generated product is the generation of undesirable secondary structures, especially between copies whose single strands are complementary to one another. In FIG. 11A, a copy of 1116 (also marked 1116) may anneal to a copy of 1108 (also marked 1108) within the RCA-generated product, rendering the copy of 1108 inaccessible to probes used during cPAL. This undesirable annealing can be prevented by the way the adaptor and the hairpin adaptor are made, with 1112 being complementary to 1104. As shown in FIG. 11A, the RCA-generated segment 1120 is identical to 1104, and the RCA-generated segment 1121 is identical to 1112. Since 1112 is complementary to 1104, 1121 anneals to 1120 as RCA proceeds, thus preventing annealing of 1116 to 1108. The entire RCA product is not shown; 1122 is part of the copied vector. After RCA is complete, cPAL anchors 1123 and 1124 can anneal to single-stranded regions of RCA construct such as 1110, and initiate sequencing towards the direction of the arrows.

1104 and 1112 are designed in a way favoring fast annealing that completes before RCA generates 1116. Those skilled in the art know how to design sequences with desired kinetics of secondary structure formation.

In another embodiment shown in FIG. 11B, adaptor and hairpin designs are such that annealing between copies of 1116 and 1108 is not prevented during RCA construction. In this case, one of the two copies is rendered single-stranded by destroying the other copy. Specifically, segment 1114 of the hairpin adaptor is at least partially complementary to segment 1110, and comprises a restriction site recognized by a nicking endonuclease. When the RCA construct is exposed to a reaction solution comprising nicking endonucleases, nick 1130 is generated. Then, the RCA construct is exposed to a reaction solution comprising 5′-3′ exonucleases (such as T7 exonuclease) that preferentially digest double-stranded DNA and can initiate digestion from the 5′ end exposed at the nick 1130 (or 5′-3′ exonucleases are included in the nicking reaction). Exonuclease-mediated destruction of 1116 exposes 1108, rendering it accessible for sequencing using cPAL or other methods. For example, anchor 1131 can bind to a digestion-exposed part of 1114 and initiate cPAL sequencing towards the direction of the arrow.

Washing and other treatments may be applied in between described steps as recognized and known by those skilled in the art.

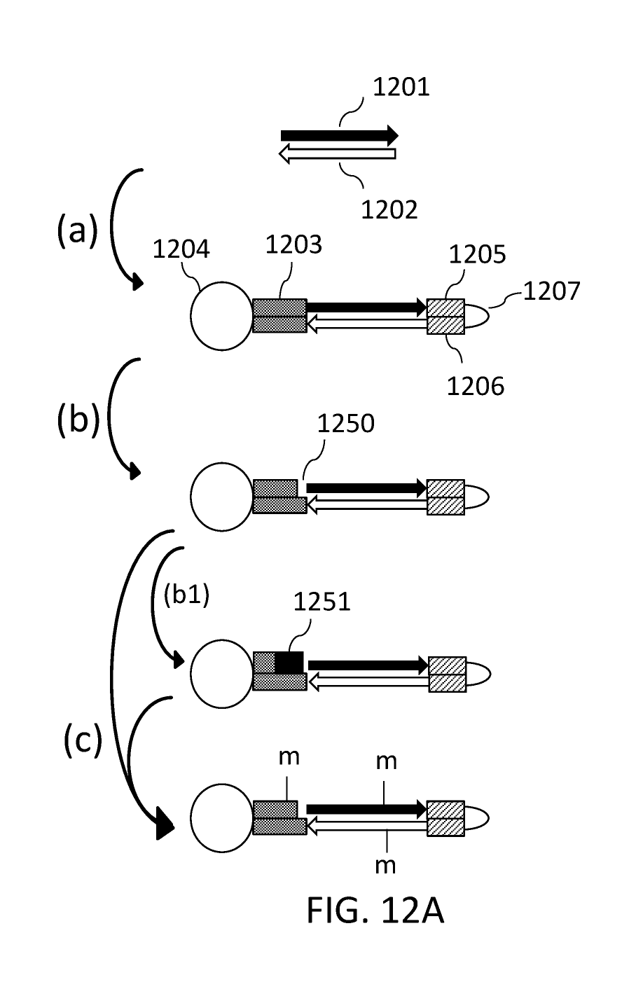

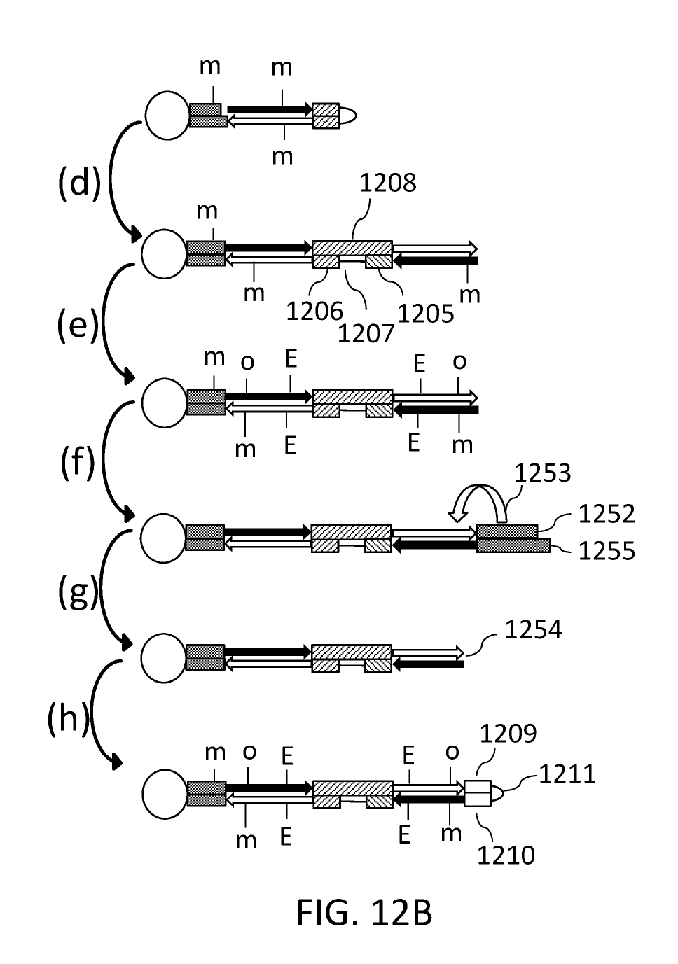

In another embodiment shown in FIG. 12A, a nucleic acid molecule is a blunt-ended double-stranded DNA molecule comprising strand 1201 and strand 1202. The two strands are represented as arrows demonstrating 5′-to-3′ orientation. DNA molecules such as this can be generated, for example, by randomly cleaving genomic DNA material, and repairing the ends of the resulting DNA fragments.

During step (a), the nucleic acid molecule is ligated to an adaptor 1203 that is anchored to the surface of a bead 1204. In other embodiments, 1203 is not anchored. 1203 is blunt in this embodiment. The other end of the nucleic acid molecule that is not ligated to 1203, is ligated to a blunt-ended hairpin adaptor comprising two at least partially complementary segments 1205 and 1206, and a loop 1207.

During step (b), the nucleic acid molecule and its surroundings are subjected to incubation with nicking restriction endonuclease molecules that recognize a specific restriction site within the adaptor 1203 and create a nick 1250 exposing the last 3′ end of 1203 (upper strand) and the first 5′ end of the nucleic acid molecule (strand 1201). In other embodiments, the nick is within 1203, or within the nucleic acid molecule. The restriction site is chosen to be recognized by methylation-sensitive nicking restriction endonucleases, such as Nt.AlwI or Nt.BstNBI. Methylation sensitivity is discussed elsewhere herein.

During step (c), the nucleic acid molecule and its surroundings are exposed to a reaction solution comprising methyltransferases. Strands and segments that may become methylated are marked with “m” in FIG. 12A. The purpose of this step is to methylate adaptor 1203 so that future nicking steps cannot cause nicking originating from the nicking endonuclease site in adaptor 1203 (methylation may occur in both strands of the adaptor, but only the upper adaptor strand is marked with “m” for simplicity). In some embodiments, 1201 and 1202 may be methylated in advance, before participating in step (a).

In some embodiments, the presence of the nick 1250 may prevent methylation of 1203 during step (c). For some enzymes, a certain number of nucleotides may be needed between the recognition site and the nearby free 3′ or 5′ end for optimal catalysis. This is at least the case for restriction enzymes, which, as a general recommendation, may prefer around 6 base pairs on either side of the recognition site (Pingoud et al., 2014); (https://www.neb.com/tools-and-resources/usage-guidelines/cleavage-close-to-the-end-of-dna-fragments). In this case, nicking is performed within the adaptor, with at least one base following the nick residing within the adaptor. During step (b1), the nucleic acid molecule and its surroundings are exposed to a polymerization reaction solution comprising polymerase molecules (which may comprise 5′-3′ exonuclease activity and/or strand-displacing activity) and nucleotides with appropriate base type or types to allow extension and replacement of the at least one base following the nick. In some related embodiments, at least some of the bases within 1203 that follow the nick form a short homopolymer sequence. For example, 6 bases following the nick within the adaptor 1203 form a homopolymer comprising cytosine. During step (b1), the polymerization reaction solution comprises only dCTPs to extend the nick by 6 bases (1251). The homopolymer may be followed by at least one base within the adaptor, which is of different type from the homopolymer (for example, A, T or G, in the event that the homopolymer has Cs), so that the extension 1251 formed during step (b1) stops within the adaptor. 1251 is long enough to ensure proper recognition of the methylase recognition site by methylases during step (c).

The hairpin adaptor comprising 1205, 1206 and 1207 is not methylated while being in its folded conformation, because it is designed so that the methylase recognition site in the hairpin adaptor comprises at least a mismatch in the folded conformation, or said methylase recognition site resides at least partially within loop 1207.

During step (d) in FIG. 12B, the nucleic acid molecule and its surroundings are exposed to conditions to cause nucleotide incorporation, and to a template-dependent polymerization reaction solution comprising nucleotides and polymerase molecules comprising strand-displacing activity. The polymerization reaction in step (d) produces segments that are complementary to the two strands 1201 and 1202 of the nucleic acid molecule, and a segment 1208 that is complementary to segment 1206 of the hairpin adaptor, loop 1207 of the hairpin adaptor, and segment 1205 of the hairpin adaptor. The newly formed segments are not methylated. The segments that are methylated are shown marked with “m” (methylation may occur in both strands of the adaptor, but only the upper adaptor strand is marked with “m” for simplicity). The product that results from this step has two copies of the nucleic acid molecule, one copy being inverted in relation to the other.

During step (e), the nucleic acid molecule and its surroundings are exposed to a reaction solution comprising methylases. These methylases specifically methylate sites within the copies of the nucleic acid molecule, to block restriction endonucleases used during the following step (g). The purpose of this step is to protect the nucleic acid molecule's copies from undesirable digestion. Potentially methylated strands are marked with “E”. Optionally, in this step, the reaction solution may also comprise methylases that specifically recognize hemimethylated sites generated during previous steps. For example, CcrM and Dnmt1 preferentially methylate the non-methylated strand of their hemimethylated recognition site. Such optional methylation is desired in the event that future nicking steps use nicking endonucleases that are not blocked by hemimethylation; full methylation in this case protects the nucleic acid molecule's copies from undesirable digestion. Optionally methylated segments are marked with “o” in FIG. 12B. Methylations during step (e) may be performed in a single reaction, or step (e) may comprise sub-steps, one for each methyltransferase type used.

The process continues with step (f) which comprises ligating an adaptor 1252. During step (g), the nucleic acid molecule and its surroundings are subjected to incubation with restriction endonuclease molecules that recognize a restriction site within 1252. These restriction endonuclease molecules cut outside of their restriction site and inside the nucleic acid molecule copy, as shown by arrow 1253. Example of such restriction endonuclease is EcoP15I.

Undesirable cutting from sites within the nucleic acid molecule's copies is prevented by methylations (“E”) generated during step (e). Step (g) produces truncated nucleic acid molecule copy 1254. The truncated copy may have a blunt end or an end with an overhang, depending on the enzyme that performs the cutting. Those skilled in the art know techniques to create an end suitable for subsequent applications such as ligation to an adaptor. For example, overhangs may be filled or chewed back to yield blunt ends, in the event that blunt ligation is desired. In another non-limiting example, a polymerase such as Taq is used to create a single-base 3′-end overhang comprising adenine, suitable for TA ligation to an adaptor.

The adaptor 1252 may have an overhang or recessive end or modification at the 3′ end 1255, which may prevent ligation of hairpin or other adaptors during future steps, in the event that enzymatic cleavage during step (g) is incomplete.

In some embodiments, steps (f) and (g) are repeated one or more times using the same or different enzymes, in the event that construction of a shorter copy is desired.

During step (h), the truncated copy 1254 is ligated to a hairpin adaptor comprising two at least partially complementary segments 1209 and 1210, and a loop 1211.

Washing and other treatments may be applied in between described steps as recognized and known by those skilled in the art.

After step (h), the process can continue by repeating steps (b) through (h) one or more times, to generate progressively truncated copies of a nucleic acid molecule. Steps (b) through (h) constitute a cycle. Nicking during step (b) of each cycle involves a restriction site in the hairpin adaptor that is attached during the step before step (b) of the previous cycle. For example, step (b) that follows step (h) of FIG. 12B can create a nick that is produced by restriction endonucleases recognizing a restriction site within 1208 of the hairpin adaptor attached during step (a). Methylation steps prevent unwanted nicking originating from the other adaptors.

Washing and other treatments may be applied in between described steps as recognized and known by those skilled in the art.

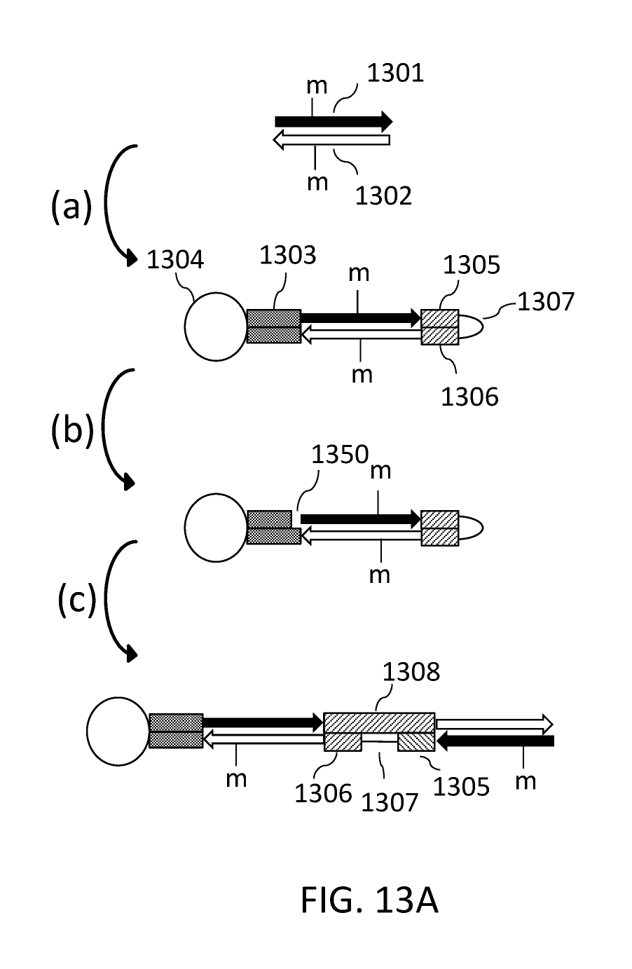

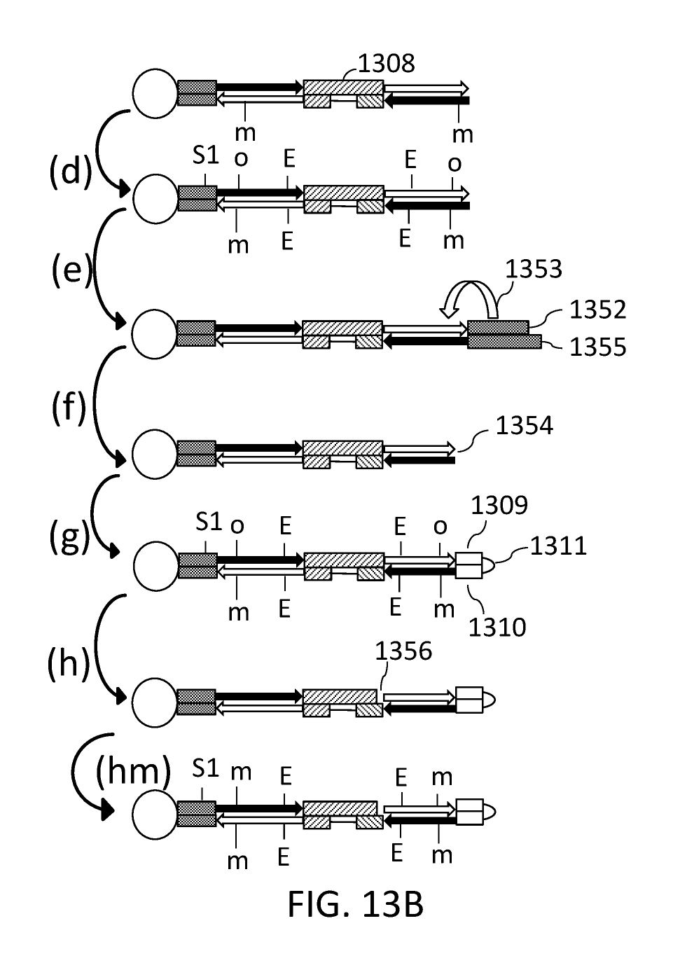

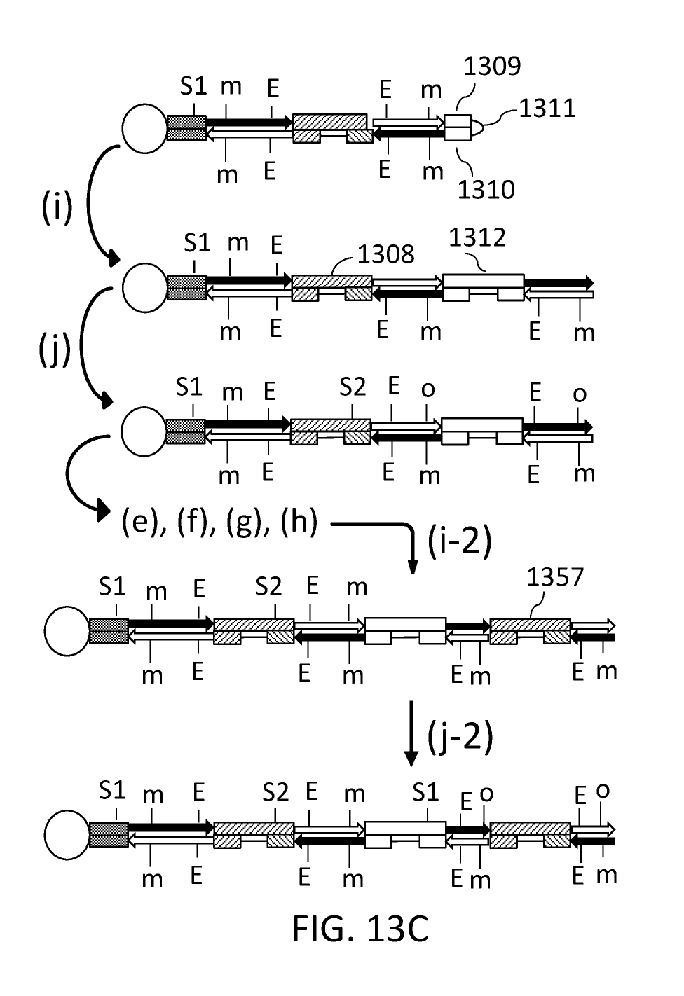

In another embodiment shown in FIG. 13A, a nucleic acid molecule is a blunt-ended double-stranded DNA molecule comprising strand 1301 and strand 1302. The two strands are represented as arrows demonstrating 5′-to-3′ orientation. DNA molecules such as this can be generated, for example, by randomly cleaving genomic DNA material, and repairing the ends of the resulting DNA fragments. The nucleic acid molecule is methylated with appropriate methyltransferases, to prevent undesirable nicking during subsequent nicking steps. After methylation, the nucleic acid molecule may be purified by phenol extraction followed by ethanol precipitation, or other methods. Methylation and purification protocols are well known to those skilled in the art. Methylated strands are labeled with “m”.

During step (a), the nucleic acid molecule is ligated to an adaptor 1303 that is anchored to the surface of a bead 1304. In other embodiments, 1303 is not anchored. 1303 is blunt in this embodiment. The other end of the nucleic acid molecule that is not ligated to 1303, is ligated to a blunt-ended hairpin adaptor comprising two at least partially complementary segments 1305 and 1306, and a loop 1307.

During step (b), the nucleic acid molecule and its surroundings are subjected to incubation with nicking restriction endonuclease molecules that recognize a specific restriction site within the adaptor 1303 and create a nick 1350 exposing the last 3′ end of 1203 (upper strand) and the first 5′ end of the nucleic acid molecule (strand 1301). In other embodiments, the nick is within 1303, or within the nucleic acid molecule. The restriction site is chosen to be recognized by methylation-sensitive nicking restriction endonucleases, such as Nt.AlwI or Nt.BstNBI. Methylation sensitivity is discussed elsewhere herein.

During step (c), the nucleic acid molecule and its surroundings are exposed to conditions to cause nucleotide incorporation, and to a template-dependent polymerization reaction solution comprising nucleotides and polymerase molecules comprising strand-displacing activity. The polymerization reaction in step (c) produces segments that are complementary to the two strands 1301 and 1302 of the nucleic acid molecule, and a segment 1308 that is complementary to segment 1306 of the hairpin adaptor, loop 1307 of the hairpin adaptor, and segment 1305 of the hairpin adaptor. The newly formed segments are not methylated. The segments that are methylated are shown marked with “m”. The product that results from this step has two copies of the nucleic acid molecule, one copy being inverted in relation to the other.

During step (d) in FIG. 13B, the nucleic acid molecule and its surroundings are exposed to a reaction solution comprising methylases. Methylases in the reaction solution specifically methylate sites within the copies of the nucleic acid molecule that can block restriction endonucleases used during the following step (f). The purpose is to protect the nucleic acid molecule's copies from undesirable digestion. Potentially methylated strands are marked with “E”. This step also comprises using methylases specific for a methylase recognition site within the adaptor, causing methylation marked with “S1”. Methylation may occur in both strands of the adaptor, but only the upper adaptor strand is marked with “S1” for simplicity. Methylation “S1” blocks any future nicking originating from the nicking endonuclease site in the adaptor. Methylation “S1” may also occur within the nucleic acid molecule's copies (not marked, for simplicity). The hairpin adaptor is designed so that 1308 does not comprise the same methylase recognition site. Optionally, in this step, the reaction solution may also comprise methylases that specifically recognize hemimethylated sites generated during previous steps. For example, CcrM and Dnmt1 preferentially methylate the non-methylated strand of their hemimethylated recognition site. Such optional methylation is desired in the event that future nicking steps use nicking endonucleases that are not blocked by hemimethylation; full methylation in this case protects the nucleic acid molecule's copies from undesirable digestion. Optionally methylated segments are marked with “o” in FIG. 13B. Methylations during step (d) may be performed in a single reaction, or step (d) may comprise sub-steps, one for each methyltransferase type used.

The process continues with step (e) which comprises ligating an adaptor 1352. During step (f), the nucleic acid molecule and its surroundings are subjected to incubation with restriction endonuclease molecules that recognize a restriction site within 1352. These restriction endonuclease molecules cut outside of their restriction site and inside the nucleic acid molecule copy, as shown by arrow 1353. Example of such restriction endonuclease is EcoP15I. Undesirable cutting from sites within the nucleic acid molecule's copies is prevented by methylations (“E”) generated during step (d). Step (f) produces truncated nucleic acid molecule copy 1354. The truncated copy may have a blunt end or an end with an overhang, depending on the enzyme that performs the cutting. Those skilled in the art know techniques to create an end suitable for subsequent applications such as ligation to an adaptor. For example, overhangs may be filled or chewed back to yield blunt ends, in the event that blunt ligation is desired. In another non-limiting example, a polymerase such as Taq is used to create a single-base 3′-end overhang comprising adenine, suitable for TA ligation to an adaptor.

The adaptor 1352 may have an overhang or recessive end or modification at the 3′ end 1355, which may prevent ligation of hairpin or other adaptors during future steps, in the event that enzymatic cleavage during step (f) is incomplete.

In some embodiments, steps (e) and (f) are repeated one or more times using the same or different enzymes, in the event that construction of a shorter copy is desired.

During step (g), the truncated copy 1354 is ligated to a hairpin adaptor comprising two at least partially complementary segments 1309 and 1310, and a loop 1311.

During step (h), the nucleic acid molecule and its surroundings are subjected to incubation with nicking restriction endonuclease molecules that recognize a specific restriction site within the hairpin adaptor (1308) and create a nick 1356 exposing the last 3′ end of 1308 (upper strand) and the first 5′ end of the nucleic acid molecule copy. In other embodiments, the nick is within 1308, or within the nucleic acid molecule copy.

During step (hm), the nucleic acid molecule and its surroundings are exposed to a reaction solution comprising methylases. Methylases methylate sites within the nucleic acid molecule copies. These methylation sites may be the same or different from the methylation sites in the adaptor and hairpin adaptor. The methylase recognition sites may be the same or different from the methylase recognition site in the adaptor. Hairpin adaptor 1308 may or may not become methylated during this step. This step ensures methylation of the upper strands, and can be omitted, in the event that the optional methylation is performed in step (d).

The hairpin adaptor comprising 1309, 1310 and 1311 is not methylated while being in its folded conformation, because it is designed and produced so that any methylase recognition site in the hairpin adaptor comprises at least a mismatch in the folded conformation, or said methylase recognition site resides at least partially within loop 1311.

During step (i) in FIG. 13C, the nucleic acid molecule and its surroundings are exposed to conditions to cause nucleotide incorporation, and to a template-dependent polymerization reaction solution comprising nucleotides and polymerase molecules comprising strand-displacing activity. The polymerization reaction in step (i) produces segments that are complementary to the two strands of the nucleic acid molecule copy, and a segment 1312 that is complementary to segment 1310 of the hairpin adaptor, loop 1311 of the hairpin adaptor, and segment 1309 of the hairpin adaptor. The newly formed segments are not methylated.

During step (j) in FIG. 13C, the nucleic acid molecule and its surroundings are exposed to a reaction solution comprising methylases. The purpose is to protect the nucleic acid molecule's copies from undesirable digestion. Potentially methylated strands are marked with “E”. This step also comprises using methylases specific for a methylase recognition site within the hairpin adaptor (1308), causing methylation marked with “S2”. Methylation “S2” blocks any future nicking originating from the nicking endonuclease site in the adaptor. Methylation may occur in both strands of the adaptor, but only the upper hairpin adaptor strand is marked with “S2” for simplicity. Methylation “S2” may also occur within the nucleic acid molecule's copies (not marked, for simplicity). The hairpin adaptor comprising 1309, 1310 and 1311 is designed so that 1312 does not comprise the same methylase recognition site. Optionally, in this step, the reaction solution may also comprise methylases that specifically recognize hemimethylated sites generated during previous steps. For example, CcrM and Dnmt1 preferentially methylate the non-methylated strand of their hemimethylated restriction site. Such optional methylation is desired in the event that future nicking steps use nicking endonucleases that are not blocked by hemimethylation; full methylation in this case protects the nucleic acid molecule's copies from undesirable digestion. Optionally methylated segments are marked with “o”. Methylations during step (j) may be performed in a single reaction, or step (j) may comprise sub-steps, one for each methyltransferase type used.

Washing and other treatments may be applied in between described steps as recognized and known by those skilled in the art.

After step (j), the process can continue by repeating steps (e) through (j) one or more times, to generate progressively truncated copies of a nucleic acid molecule. Steps (e) through (j) constitute a cycle. The hairpin adaptor 1357 attached during the second cycle has a methylase recognition site different from the hairpin adaptor 1312, and may have the same methylase recognition site with the hairpin adaptor 1308. During step (j-2) of the second cycle, hairpin adaptor 1312 is methylated. This hairpin adaptor can be methylated with the same type of methylase as the first adaptor 1303 (“S1”). The hairpin adaptor attached in one cycle can have a methylase recognition site of the same type (“S1” or “S2”) with that of the hairpin adaptor attached in the cycle before the previous.

Washing and other treatments may be applied in between described steps as recognized and known by those skilled in the art.

In one example related to the embodiment described in FIGS. 13A-C, adaptor 1303 and the hairpin adaptor 1312 comprise the sequence GGATCC. The part GGATC is recognized by Nt.AlwI, the part GATC is recognized by (i.e. is a methylase recognition site for) adenine methyltransferases such as dam methyltransferase, whereas the entire GGATCC sequence is a methylase recognition site for BamHI methyltransferase. The methylation site for dam methyltransferase is the A in GATC, whereas the methylation site for BamHI methyltransferase is the first C in the GGATCC sequence. Methylated sequences G-mA-CT and GGAT-mC-C block Nt.AlwI, preventing nicking by this endonuclease (McClelland et al., 1994). Steps (d) and (j-2) comprise using BamHI methyltransferase to methylate adaptor 1303 and hairpin adaptor 1312 respectively (methylations marked with “S1”). Hairpin adaptor 1308 comprises the sequence GGATCG. The part GGATC is recognized by Nt.AlwI, the part GATC is recognized by dam methyltransferase, whereas the entire GGATCG sequence is a methylase recognition site for M.SssI. The methylation site for M.SssI is the same base within the Nt.AlwI site as in the case of BamHI methyltransferase (the C), and, similarly, the M.SssI-methylated sequence GGAT-mC-G blocks Nt.AlwI. Step (j) comprises using M.SssI to methylate hairpin adaptor 1308 (methylation marked with “S2”). Step (hm) comprises using dam methyltransferase or other related enzymes, to methylate any Nt.AlwI sites within the nucleic acid molecule copies. The nucleic acid molecule (1301, 1302) may also be methylated by dam methyltransferase (marked “m”).

In another example related to the embodiment described in FIGS. 13A-C, adaptor 1303 and the hairpin adaptor 1312 comprise the sequence TCTAGAGTC. The part GAGTC is recognized by Nt.BstNBI and the HinfI methyltransferase (which recognizes GANTC), and the part TCTAGA is recognized by (i.e. is a methylase recognition site for) M.XbaI methyltransferase. The methylation site for both HinfI and M.XbaI methyltransferases is the A in GAGTC. Methylated sequence G-mA-GTC blocks Nt.BstNBI, preventing nicking by this endonuclease. Steps (d) and (j-2) comprise using M.XbaI methyltransferase to methylate adaptor 1303 and hairpin adaptor 1312 respectively (methylations marked with “S1”). Hairpin adaptor 1308 comprises the sequence TCGAGTC. The part GAGTC is recognized by Nt.BstNBI and the HinfI methyltransferase (which recognizes GANTC), and the part TCGA is recognized by (i.e. is a methylase recognition site for) TaqI methyltransferase. The methylation site for TaqI methyltransferase is also A, leading to the same G-mA-GTC sequence that blocks Nt.BstNBI. Step (j) comprises using TaqI methyltransferase to methylate hairpin adaptor 1308 (methylation marked with “S2”). Step (hm) comprises using HinfI methyltransferase to methylate any Nt.BstNBI sites within the nucleic acid molecule copies. The nucleic acid molecule (1301, 1302) may also be methylated by HinfI methyltransferase (marked “m”).

In certain embodiments related to the one described in FIGS. 13A-C, methylation reactions (not including the “E” type) in step (d) can be performed after step (e), or after step (f) or after step (g).

In some embodiments, methyltransferases are not used. Instead, nucleic acid molecules are pre-treated with restriction endonucleases that destroy the recognition sites of the nicking endonucleases to be used when constructing consecutive copies of said nucleic acid molecules. For example, in the event that Nt.AlwI is to be used for nicking, pre-treatment with MboI cuts at the GATC site within the Nt.AlwI restriction site.

In some embodiments, producing methylations type “E” and steps (e) and (f) are omitted, so that the generated nucleic acid molecule copies are not truncated.

Another method of producing consecutively connected copies of nucleic acid molecules is shown in

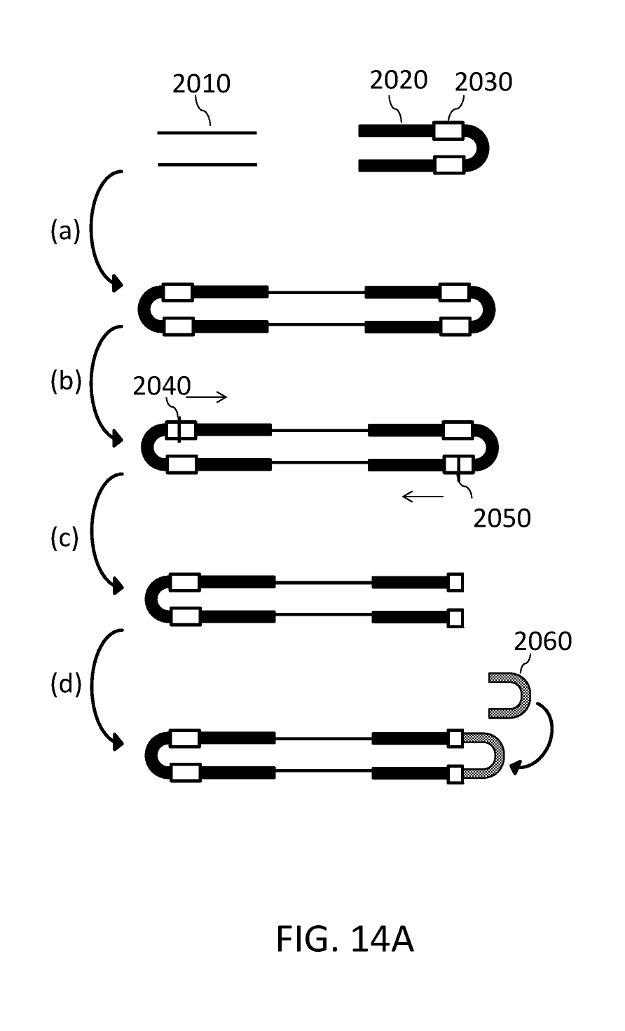

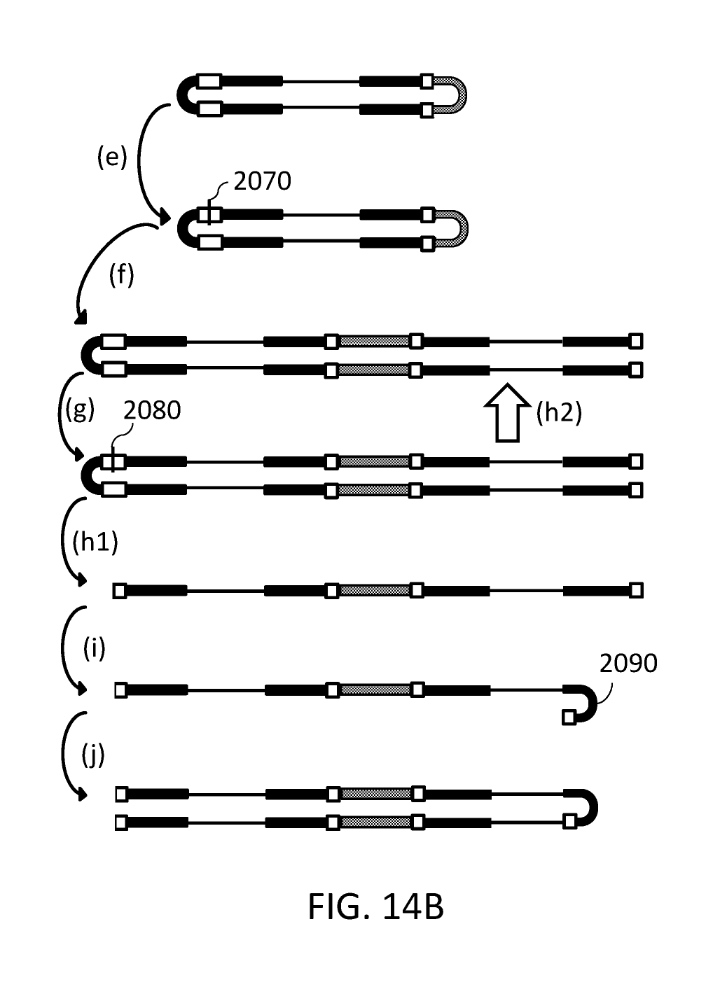

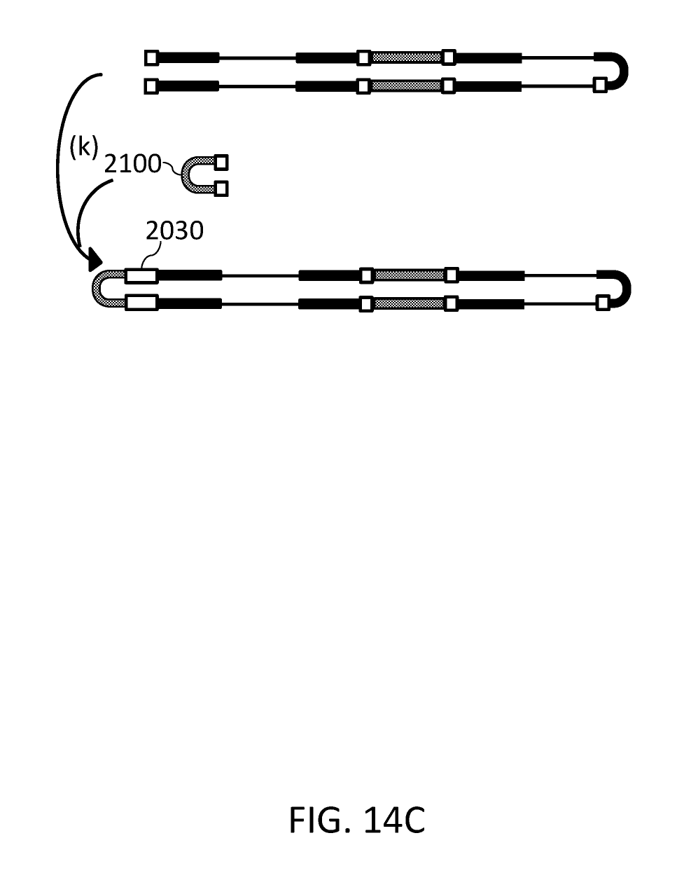

In another embodiment shown in FIG. 14A, a blunt-ended nucleic acid molecule 2010 is ligated to hairpins 2020 comprising nicking endonuclease recognition sites 2030 during step (a). During step (b), the circularized molecule generated in step (a) is exposed to nicking endonucleases which create nicks 2040 and 2050 within the 2030 restriction sites. During step (c), the 3′ ends at the nicks are extended towards the direction of the arrows by using strand-displacing polymerases, generating long hairpin constructs. Each circularized molecule can yield two such long hairpin constructs during this step.

During step (d), hairpin adaptors 2060 are ligated to the free ends of the long hairpin constructs from the previous step, generating another circularized molecule. As hairpins 2060 do not comprise nicking endonuclease sites, the newly generated circularized molecule has only one nicking endonuclease site.

As shown in FIG. 14B, during step (e), the circularized molecule from step (d) is exposed to nicking endonucleases generating nick 2070.

During step (f), the 3′ end at the 2070 nick can be extended by using strand-displacing polymerases, forming a long hairpin construct which comprises another copy of the original nucleic acid molecule 2010.

During step (g), nicking endonucleases create nick 2080 (which, in essence, is the nick 2070 regenerated).

During step (h), strand-displacing extension from the 3′ end of the nick 2080 regenerates the long hairpin construct in step (g) [step (h2)] and displaces a single strand [step (h1)]. Repeated hairpin regeneration and nicking can create multiple displaced single strands, thus amplifying the material.

During step (i), the 3′ end of the displaced single strand from step (h1) can form a hairpin with an extendable 3′ end, in the event that hairpin 2020 is designed in such a way so that part of its sequence comprising part of the nicking site exhibits at least partial self-complementarity.

During step (j), extension from the extendable 3′ end from the previous step forms a new long hairpin construct.

As shown in FIG. 14C, during step (k), the new long hairpin construct can be ligated to a hairpin 2100 which comprises part of the nicking endonuclease recognition site, so that ligation during this step regenerates the full length of the nicking endonuclease recognition site. The newly formed circularized construct can participate in a cycle comprising steps (e) through (k). This cycle can be repeated several times, with circularized molecules generated during step (k) comprising a number of copies of the original 2010 nucleic acid molecule that is double the number present in the constructs participating in step (e).

The cycle comprising steps (e) through (k) can be repeated multiple times and performed with at least two steps conducted separately, or at least two step conducted in the same reaction solution. For example, all steps can be conducted in the same reaction solution, with nicking and extension conducted at a specific temperature that does not favor ligation, followed by incubation at a temperature that favors ligation but not nicking and extension.

### Example 1: Generation of Consecutively Connected Copies

### Step 1: Preparation of Hairpin Adaptors (“Hairpin_Nt”) Comprising a Site for the Nicking Endonuclease Nt.BbvCI.

Those skilled in the art can design hairpin oligonucleotides and synthesize them with standard methods. A part of a hairpin adaptor may be designed so that it is a random sequence, which can serve as an identifier. For example, an oligonucleotide can be synthesized so that a random sequence is placed at its 5′ end. The oligonucleotide is designed so that it can form a hairpin with the random sequence being a 5′ end overhang. After hairpin formation, the 3′ end of the oligonucleotide can be extended appropriately to form an end that can participate in future ligation steps.

Hairpin_Nt is a hairpin adaptor comprising a site for Nt.BbvCI, and also comprises a biotinylated thymine inside its loop that enables binding to streptavidin-coated magnetic beads.

First, 1 μl of Hairpin_Nt (100 μM stock) is added to 200 μl of Annealing Buffer (10 mM Tris pH 7.5, 100 mM NaCl) and incubated at 95° C. for 10 min, then gradually cooled down to room temperature, to promote proper self-annealing of the hairpins.

Then, annealed Hairpin_Nt hairpins are bound to streptavidin-coated beads: 100 μl of Dynabeads® magnetic beads (1 mg/100 μl; Thermo Fisher Scientific) are added to 1 ml 1×BW buffer (5 mM Tris-HCl (pH 7.5), 0.5 mM EDTA, 1 M NaCl) and resuspended, then placed on a magnet for 1 min and the supernatant is discarded. The sample is removed from the magnet and the beads are resuspended in 100 μl of 2×BW buffer (10 mM Tris-HCl (pH 7.5), 1 mM EDTA, 2 M NaCl). Washing (first 1×BW, then 2×BW) is repeated for a total number of three washes. In order to bind Hairpin_Nt to the beads, 200 μl of 2×BW is added to the washed beads, and the 200 μl of annealed hairpins are added to the mix. The mix is incubated for 15 min at room temperature using gentle rotation. Then, the sample is placed on a magnet for 2-3 min, and washed 2-3 times with 0.5 ml of 1×BW buffer. The beads are further washed three times using 0.5 ml of 1×T4 DNA Ligase reaction buffer (New England BioLabs; 50 mM Tris-HCl, 10 mM MgCl2, 1 mM ATP, 10 mM DTT) and placed on magnet to retrieve a bead pellet for further processing.

### Step 2: Ligation of Fragmented Genomic DNA.

Genomic DNA can be prepared and fragmented to desired sizes according to methods well known to those skilled in the art. For example, DNA can be fragmented to a range of 0.5-5 kb.

A reaction comprising 1 μl of fragmented genomic DNA (final 0.1 μM), 10 μl of 10×T4 DNA Ligase reaction buffer and 5 μl of T4 DNA Ligase (400,000 units/ml; New England BioLabs) is added to the washed bead pellet and incubated at room temperature (20-25° C.) for 10 minutes, to promote ligation of the DNA to Hairpin_Nt on the beads. Afterwards, the beads are washed three times using 0.5 ml of 1×NEBuffer 4 (New England BioLabs; 50 mM Potassium Acetate, 20 mM Tris-acetate, 10 mM Magnesium Acetate, 1 mM DTT) and placed on magnet to retrieve a bead pellet for further processing.

### Step 3: Preparation of Hairpin Adaptors.

The use of hairpin adaptors helps generate consecutively connected copies of nucleic acid molecules as described in detail elsewhere herein. Those skilled in the art can design hairpin oligonucleotides and synthesize them with standard methods.

10 μl of hairpin adaptors are added to a final volume of 100 μl 1×NEBuffer 4 (to a final 10 μM), and incubated at 95° C. for 10 min, then gradually cooled down to room temperature, to promote proper self-annealing of the hairpins.

### Step 4: Generation of Consecutively Connected Copies of Genomic DNA Fragments.

Consecutively connected copies of genomic DNA fragments can be constructed by performing hairpin adaptor ligation, nicking and polymerization in a single reaction. The washed bead pellet from the previous step is resuspended in a reaction comprising hairpin adaptors, 1×NEBuffer 4, 1×BSA, ATP, dNTP, phi29 DNA polymerase, Nt.BbvCI, and T4 DNA ligase. Optionally, Klenow fragment (minus 3′-5′ exonuclease) can be used, in the event that dA overhangs are desired, in order to ligate to hairpin adaptors having dT overhangs.

In another example, one or more steps (hairpin adaptor ligation, nicking, polymerization, optional A-tailing) can be carried out as different reactions. Protocols for performing ligation, nicking and polymerization are well known to those skilled in the art and readily available by reagent providers such as New England BioLabs.

### Example 2: Generation of Truncated Copies

A copy generated after a round of hairpin adaptor ligation, nicking and polymerization can be truncated using appropriate enzymes such as EcoP15I.

First, the bead pellets from the previous example can be exposed to a ligation reaction solution comprising adaptors, according to standard ligation protocols. Adaptors ligate to copies of genomic DNA fragments generated according to the previous example. Each adaptor comprises a restriction site for EcoP15I that is appropriately positioned to allow EcoP15I to cut a 27-base fragment from the copy ligated to the adaptor.

Then, after washing, the bead pellet can be resuspended in a 200 μl reaction comprising 20 μl 10×NEBuffer 3 (New England BioLabs; 1×NEBuffer: 100 mM NaCl, 50 mM Tris-HCl, 10 mM MgCl2, 1 mM DTT), 2 μl 100×BSA (10 mg/ml), 2 μl 10 mM Sinefungin, 40 μl 10 mM ATP and 1.7 μl EcoP15I (2 u/μl). The reaction is incubated at 37° C. for 2 hours.

Optionally, a methylation step can be applied prior to adaptor ligation. Specifically, the bead pellet can be resuspended in a reaction comprising NEBuffer 3, BSA, EcoP15I and SAM (S-adenosyl-methionine) based on protocols well known to those skilled in the art. This methylation step accomplishes methylation of any EcoP15I sites present within the genomic DNA fragments and their copies, thus preventing any undesirable cutting by EcoP15I in the above-described reaction.

All the methods disclosed and claimed herein may comprise washing steps, reagent exchange steps and other treatments in between described steps as recognized and known by those skilled in the art.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations can be applied to the compositions and methods and in the steps or in the sequence of steps of the methods described herein without departing from the concept, spirit and scope of the disclosure. More specifically, it will be apparent that certain agents which are both chemically related can be substituted for the agents described herein while the same or similar results would be achieved. All such similar substitutes and modifications apparent to those skilled in the art are deemed to be within the spirit, scope and concept of the disclosure as defined by the appended claims.

### REFERENCES

Barnes, C., Earnshaw, D. J., Liu, X., Milton, J., Ost, T. W. B., Rasolonjatovo, I. M. J., Rigatti, R., Romieu, A., Smith, G. P., Turcatti, G., Worsley, G. J., Wu, X., 2007. Preparation of templates for nucleic acid sequencing. WO2007010251 A3.
Beaucage, S. L., Iyer, R. P., 1993. The Functionalization of Oligonucleotides Via Phosphoramidite Derivatives. Tetrahedron 49, 1925-1963. doi:10.1016/50040-4020(01)86295-5
Benner, S. A., 1993. Oligonucleotide analogs containing sulfur linkages. U.S. Pat. No. 5,216,141 A.
Ben Yehezkel, T., Linshiz, G., Buaron, H., Kaplan, S., Shabi, U., Shapiro, E., 2008. De novo DNA synthesis using single molecule PCR. Nucleic Acids Res. 36, e107. 1590 doi:10.1093/nar/gkn457
Berg, J. L. T. L. S. J., 2006. Biochemistry 6th Edition (Sixth Ed.) 6e By Jeremy Berg, John Tymoczko & Lubert Stryer 2006. Example Product Manufacturer.
Bridgham, J., Corcoran, K., Golda, G., Pallas, M. C., Brenner, S., 2013. System and apparatus for sequential processing of analytes. US20130184162 A1.
Brill, W. K. D., Tang, J. Y., Ma, Y. X., Caruthers, M. H., 1989. Synthesis of oligodeoxynucleoside phosphorodithioates via thioamidites. J. Am. Chem. Soc. 111, 2321-2322. doi:10.1021/ja00188a066
Buermann, D., Moon, J. A., Crane, B., Wang, M., Hong, S. S., Harris, J., Hage, M., Nibbe, M. J., 2013. Integrated optoelectronic read head and fluidic cartridge useful for nucleic acid sequencing. US20130260372 A1.
Carlsson, C., Jonsson, M., Nordén, B., Dulay, M. T., Zare, R. N., Noolandi, J., Nielsen, P. E., Tsui, L.-C., Zielenski, J., 1996. Screening for genetic mutations. Nature 380, 207-207. doi:10.1038/380207a0
Casadesús, J., Low, D., 2006. Epigenetic Gene Regulation in the Bacterial World. Microbiol. Mol. Biol. Rev. 70, 830-856. doi:10.1128/MMBR.00016-06
Chee, M., Cronin, M. T., Fodor, S. P. A., Huang, X. X., Hubbell, E. A., Lipshutz, R. J., Lobban, P. E., Morris, M. S., Sheldon, E. L., 1998. Arrays of nucleic acid probes on biological chips. U.S. Pat. No. 5,837,832 A.
Chiu, R. W. K., Cantor, C. R., Lo, Y. M. D., 2009. Non-invasive prenatal diagnosis by single molecule counting technologies. Trends Genet. TIG 25, 324-331. doi:10.1016/j.tig.2009.05. 004
Cook, P. D., Acevedo, O., Hebert, N., 1997. Phosphoramidate and phosphorothioamidate oligomeric compounds. U.S. Pat. No. 5,637,684 A.
Cook, P. D., Sanghvi, Y. S., 1992. Nuclease resistant, pyrimidine modified oligonucleotides that detect and modulate gene expression. WO1992002258 A1.
Crawford, M. L., White, J., 2015. Method for characterising a double stranded nucleic acid using a nano-pore and anchor molecules at both ends of said nucleic acid. WO2015150786 A1.
Dean, F. B., Nelson, J. R., Giesler, T. L., Lasken, R. S., 2001. Rapid Amplification of Plasmid and Phage DNA Using Phi29 DNA Polymerase and Multiply-Primed Rolling Circle Amplification. Genome Res. 11, 1095-1099. doi:10.1101/gr.180501
De Mesmaeker, A., Waldner, A., S. Sanghvi, Y., Lebreton, J., 1994. Comparison of rigid and flexible backbones in antisense oligonucleotides. Bioorg. Med. Chem. Lett. 4, 395-398. doi:10.1016/0960-894X(94)80003-0
Dempcy, R. O., Browne, K. A., Bruice, T. C., 1995. Synthesis of a thymidyl pentamer of deoxyribonucleic guanidine and binding studies with DNA homopolynucleotides. Proc. Natl. Acad. Sci. 92, 6097-6101.
Dieffenbach, C. W., Dveksler, G. S., 2003. PCR Primer: A Laboratory Manual, 2 Lab edition. ed. Cold Spring Harbor Laboratory Press, Cold Spring Harbor, N.Y.
Drmanac, R., Callow, M., 2013. Nucleic acid sequencing and process. U.S. Pat. No. 8,518,640 B2.
Drmanac, R., Sparks, A. B., Callow, M. J., Halpern, A. L., Burns, N. L., Kermani, B. G., Carnevali, P., Nazarenko, I., Nilsen, G. B., Yeung, G., Dahl, F., Fernandez, A., Staker, B., Pant, K. P., Baccash, J., Borcherding, A. P., Brownley, A., Cedeno, R., Chen, L., Chernikoff, D., Cheung, A., Chirita, R., Curson, B., Ebert, J. C., Hacker, C. R., Hartlage, R., Hauser, B., Huang, S., Jiang, Y., Karpinchyk, V., Koenig, M., Kong, C., Landers, T., Le, C., Liu, J., McBride, C. E., Morenzoni, M., Morey, R. E., Mutch, K., Perazich, H., Perry, K., Peters, B. A., Peterson, J., Pethiyagoda, C. L., Pothuraju, K., Richter, C., Rosenbaum, A. M., Roy, S., Shafto, J., Sharanhovich, U., Shannon, K. W., Sheppy, C. G., Sun, M., Thakuria, J. V., Tran, A., Vu, D., Zaranek, A. W., Wu, X., Drmanac, S., Oliphant, A. R., Banyai, W. C., Martin, B., Ballinger, D. G., Church, G. M., Reid, C. A., 2010. Human genome sequencing using unchained base reads on self-assembling DNA nanoarrays. Science 327, 78-81. doi:10.1126/science.1181498
Eckstein, F. (Ed.), 1992. Oligonucleotides and Analogues: A Practical Approach. Oxford University Press, Oxford; New York.
Edwards, J., 2012. Polony sequencing methods. US20120270740 A1.
Efcavitch, J. W., Thompson, J. F., 2010. Single-molecule DNA analysis. Annu. Rev. Anal. Chem. Palo Alto Calif. 3, 109-128. doi:10.1146/annurev.anchem.111808.073558
Egholm, M., Buchardt, O., Christensen, L., Behrens, C., Freier, S. M., Driver, D. A., Berg, R. H., Kim, S. K., Norden, B., Nielsen, P. E., 1993. PNA hybridizes to complementary oligonucleotides obeying the Watson-Crick hydrogen-bonding rules. Nature 365, 566-568. doi:10.1038/365566a0
Egholm, M., Buchardt, O., Nielsen, P. E., Berg, R. H., 1992. Peptide nucleic acids (PNA). Oligonucleotide analogs with an achiral peptide backbone. J. Am. Chem. Soc. 114, 1895-1897. doi:10.1021/ja00031a062
Fodor, S. P. A., Stryer, L., Read, J. L., Pirrung, M. C., 1998. Arrays of materials attached to a substrate. U.S. Pat. No. 5,744,305 A.
Gait, M. J. (Ed.), 1984. Oligonucleotide Synthesis: A Practical Approach. Oxford University Press, Oxford Oxfordshire; Washington, D.C.
Gao, X., Jeffs, P. W., 1994. Unusual conformation of a 3′-thioformacetal linkage in a DNA duplex. J. Biomol. NMR 4, 17-34. doi:10.1007/BF00178333
Goodwin, S., Gurtowski, J., Ethe-Sayers, S., Deshpande, P., Schatz, M., McCombie, W. R., 2015. Oxford Nanopore Sequencing and de novo Assembly of a Eukaryotic Genome. bioRxiv 013490. doi:10.1101/013490
Gormley, N. A., Smith, G. P., Bentley, D., Rigatti, R., Luo, S., 2010. Used in solid-phase nucleic acid amplification; producing template polynucleotides that have common sequences at their 5′ ends and at their 3′ ends. U.S. Pat. No. 7,741,463 B2.
Green, E. D., 1997. Genome Analysis: A Laboratory Manual. Cold Spring Harbor Laboratory Press.
Green, M. R., Sambrook, J., 2012. Molecular Cloning: A Laboratory Manual (Fourth Edition): Three-volume set, 4th edition. ed. Cold Spring Harbor Laboratory Press, Avon, Mass.
Guo, L. H., Wu, R., 1982. New rapid methods for DNA sequencing based in exonuclease III digestion followed by repair synthesis. Nucleic Acids Res. 10, 2065-2084.
Gutjahr, A., Xu, S., 2014. Engineering nicking enzymes that preferentially nick 5-methylcytosine-modified DNA. Nucleic Acids Res. gku192. doi:10.1093/nar/gku192
Harris, T. D., 2010. Enhancing resolution of sequence analysis of short DNA stretches via defined length spacers; genetic mapping and genomics. U.S. Pat. No. 7,767,400 B2.
Hart, C., Lipson, D., Ozsolak, F., Raz, T., Steinmann, K., Thompson, J., Milos, P. M., 2010. Single-molecule sequencing: sequence methods to enable accurate quantitation. Methods Enzymol. 472, 407-430. doi:10.1016/S0076-6879(10)72002-4
Heiter, D. F., Lunnen, K. D., Wilson, G. G., 2005. Site-specific DNA-nicking mutants of the heterodimeric restriction endonuclease R.BbvCI. J. Mol. Biol. 348, 631-640. doi:10.1016/j.jmb.2005.02.034
Heron, A., BROWN, C., BOWEN, R., White, J., Turner, D. J., LLOYD, J. H., YOUD, C. P., 2015. Method for attaching one or more polynucleotide binding proteins to a target polynucleotide. WO2015110813 A1.
Higgins, L. S., Besnier, C., Kong, H., 2001. The nicking endonuclease N.BstNBI is closely related to Type IIs restriction endonucleases MlyI and PleI. Nucleic Acids Res. 29, 2492-2501.
Horn, T., Chaturvedi, S., Balasubramaniam, T. N., Letsinger, R. L., 1996. Oligonucleotides with alternating anionic and cationic phosphoramidate linkages: Synthesis and hybridization of stereo-uniform isomers. Tetrahedron Lett. 37, 743-746. doi:10.1016/0040-4039(95)02309-7
Innis, M. A., Myambo, K. B., Gelfand, D. H., Brow, M. A., 1988. DNA sequencing with Thermus aquaticus DNA polymerase and direct sequencing of polymerase chain reaction-amplified DNA. Proc. Natl. Acad. Sci. U.S.A. 85, 9436-9440.
Jenkins, G. N., Turner, N. J., 1995. The biosynthesis of carbocyclic nucleosides. Chem. Soc. Rev. 24, 169-176. doi:10.1039/CS9952400169
Joos, B., Kuster, H., Cone, R., 1997. Covalent Attachment of Hybridizable Oligonucleotides to Glass Supports. Anal. Biochem. 247, 96-101. doi:10.1006/abio.1997.2017
Jung, P. M., Histand, G., Letsinger, R. L., 1994. Hybridization of Alternating Cationic/Anionic Oligonucleotides to RNA Segments. Nucleosides Nucleotides 13, 1597-1605. doi:10.1080/15257779408012174
Khandjian, E. W., 1986. UV crosslinking of RNA to nylon membrane enhances hybridization signals. Mol. Biol. Rep. 11, 107-115.
Kornberg, A., Baker, T. A., 2005. DNA Replication. University Science Books.
Koshkin, A. A., Nielsen, P., Meldgaard, M., Rajwanshi, V. K., Singh, S. K., Wengel, J., 1998. LNA (Locked Nucleic Acid): An RNA Mimic Forming Exceedingly Stable LNA:LNA Duplexes. J. Am. Chem. Soc. 120, 13252-13253. doi:10.1021/ja9822862
Letsinger, R. L., Bach, S. A., Eadie, J. S., 1986. Effects of pendant groups at phosphorus on binding properties of d-ApA analogues. Nucleic Acids Res. 14, 3487-3499. doi:10.1093/nar/14.8.3487
Letsinger, R. L., Mungall, W. S., 1970. Nucleotide chemistry. XVI. Phosporamidate analogs of oligonucleotides. J. Org. Chem. 35, 3800-3803. doi:10.1021/jo00836a048
Letsinger, R. L., Singman, C. N., Histand, G., Salunkhe, M., 1988. Cationic oligonucleotides. J. Am. Chem. Soc. 110, 4470-4471. doi:10.1021/ja00221a089
Loeb, L. A., Hood, L., Suzuki, M., 2002. Thermostable polymerases having altered fidelity and method of identifying and using same. U.S. Pat. No. 6,395,524 B2.
Mag, M., Silke, L., Engels, J. W., 1991. Synthesis and selective cleavage of an oligodeoxynucleotide containing a bridged internucleotide 5′-phosphorothioate linkage. Nucleic Acids Res. 19, 1437-1441. doi:10.1093/nar/19.7.1437
Ma, P. N.-T., 2013. Methods, compositions, and kits for amplifying and sequencing polynucleotides. U.S. Pat. No. 8,486,627 B2.
Mayer, P., Farinelli, L., Kawashima, E. H., 2013. Method of nucleic acid amplification. U.S. Pat. No. 8,476,044 B2.
McClelland, M., Nelson, M., Raschke, E., 1994. Effect of site-specific modification on restriction endonucleases and DNA modification methyltransferases. Nucleic Acids Res. 22, 3640-3659.
Meier, C., Engels, J. W., 1992. Peptide Nucleic Acids (PNAs)—Unusual Properties of Nonionic Oligonucleotide Analogues. Angew. Chem. Int. Ed. Engl. 31, 1008-1010. doi:10.1002/anie.199210081
Mesmaeker, A. D., Lebreton, J., Waldner, A., Cook, P. D., 1997. Backbone modified oligonucleotide analogs. U.S. Pat. No. 5,602,240 A.
Metzker, M. L., 2010. Sequencing technologies—the next generation. Nat. Rev. Genet. 11, 31-46. doi:10.1038/nrg2626
Morgan, R. D., Calvet, C., Demeter, M., Agra, R., Kong, H., 2000. Characterization of the specific DNA nicking activity of restriction endonuclease N.BstNBI. Biol. Chem. 381, 1123-1125. doi:10.1515/BC.2000.137
Murphy, J., Mahony, J., Ainsworth, S., Nauta, A., van Sinderen, D., 2013. Bacteriophage Orphan DNA Methyltransferases: Insights from Their Bacterial Origin, Function, and Occurrence. Appl. Environ. Microbiol. 79, 7547-7555. doi:10.1128/AEM.02229-13
Nelson, D. L., Cox, M. M., 2012. Lehninger Principles of Biochemistry, Sixth Edition edition. ed. W.H. Freeman, New York.
Nelson, M., McClelland, M., 1991. Site-specific methylation: effect on DNA modification methyltransferases and restriction endonucleases. Nucleic Acids Res. 19, 2045-2071.
Nelson, M., McClelland, M., 1987. The effect of site-specific methylation on restriction-modification enzymes. Nucleic Acids Res. 15, r219-r230.
Oroskar, A. A., Rasmussen, S. E., Rasmussen, H. N., Rasmussen, S. R., Sullivan, B. M., Johansson, A., 1996. Detection of immobilized amplicons by ELISA-like techniques. Clin. Chem. 42, 1547-1555.
Patel, P. H., Loeb, L. A., 2003. Mutant enzymatic protein for use as tool in human therapeutics and diagnostics. U.S. Pat. No. 6,602,695 B2.
Patel, P. H., Loeb, L. A., 2001. A mutant polymerase having asptyrserglnilegluleuarg amino acid sequence in the active site and possesses altered fidelity or altered catalytic activity. U.S. Pat. No. 6,329,178 B1.
Peters, B. A., Kermani, B. G., Sparks, A. B., Alferov, O., Hong, P., Alexeev, A., Jiang, Y., Dahl, F., Tang, Y. T., Haas, J., Robasky, K., Zaranek, A. W., Lee, J.-H., Ball, M. P., Peterson, J. E., Perazich, H., Yeung, G., Liu, J., Chen, L., Kennemer, M. I., Pothuraju, K., Konvicka, K., Tsoupko-Sitnikov, M., Pant, K. P., Ebert, J. C., Nilsen, G. B., Baccash, J., Halpern, A. L., Church, G. M., Drmanac, R., 2012. Accurate whole-genome sequencing and haplotyping from 10 to 20 human cells. Nature 487, 190-195. doi:10.1038/nature11236
Pierceall, W., Steinmann, K., Causey, M., Raz, T., Jarosz, M., Buzby, P., Thompson, J., 2010. Methods of sample preparation for nucleic acid analysis for nucleic acids available in limited amounts. WO2010048386 A1.
Pingoud, A., Wilson, G. G., Wende, W., 2014. Type II restriction endonucleases—a historical perspective and more. Nucleic Acids Res. gku447. doi:10.1093/nar/gku447
Quake, S., 2011. Methods and kits for analyzing polynucleotide sequences. U.S. Pat. No. 7,981,604 B2.
Raghavendra, N. K., Rao, D. N., 2005. Exogenous AdoMet and its analogue sinefungin differentially influence DNA cleavage by R.EcoP15I—usefulness in SAGE. Biochem. Biophys. Res. Commun. 334, 803-811. doi:10.1016/j.bbrc.2005.06.171
RAWLS, R. L., 1997. OPTIMISTIC ABOUT ANTISENSE. Chem. Eng. News Arch. 75, 35-39. doi:10.1021/cen-v075n022.p035
Rigatti, R., Ost, T. W. B., 2010. Method for pair-wise sequencing a plurity of target polynucleotides. U.S. Pat. No. 7,754,429 B2.
Samuelson, J. C., Zhu, Z., Xu, S., 2004. The isolation of strand-specific nicking endonucleases from a randomized SapI expression library. Nucleic Acids Res. 32, 3661-3671. doi:10.1093/nar/gkh674
Sanghvi, Y. S., Cook, P. D. (Eds.), 1994. Carbohydrate Modifications in Antisense Research. American Chemical Society, Washington, D.C.
Sawai, H., 1984. SYNTHESIS AND PROPERTIES OF OLIGOADENYLIC ACIDS CONTAINING 2&prime;-5&prime; PHOSPHORAMIDE LINKAGE. Chem. Lett. 13, 805-808. doi:10.1246/c1.1984.805
Schleifer, A., Tom-Moy, M., 2000. Coupling linking agent to end of full-length oligonucleotides in mixture of variable length synthesized oligonucleotides, cleaving other end from support, depositing mixture on surface, linking group preferentially attaches to surface. U.S. Pat. No. 6,077,674 A.
Schmidt, V. K., Sørensen, B. S., Sørensen, H. V., Alsner, J., Westergaard, O., 1994. Intramolecular and intermolecular DNA ligation mediated by topoisomerase II. J. Mol. Biol. 241, 18-25. doi:10.1006/jmbi.1994.1469
Shuga, J., Zeng, Y., Novak, R., Lan, Q., Tang, X., Rothman, N., Vermeulen, R., Li, L., Hubbard, A., Zhang, L., Mathies, R. A., Smith, M. T., 2013. Single molecule quantitation and sequencing of rare translocations using microfluidic nested digital PCR. Nucleic Acids Res. 41, e159. doi:10.1093/nar/gkt613
Smith, S. B., Finzi, L., Bustamante, C., 1992. Direct mechanical measurements of the elasticity of single DNA molecules by using magnetic beads. Science 258, 1122-1126. doi:10.1126/science.1439819
Sprinzl, M., Sternbach, H., Von Der Haar, F., Cramer, F., 1977. Enzymatic Incorporation of ATP and CTP Analogues into the 3′ End of tRNA. Eur. J. Biochem. 81, 579-589. doi:10.1111/j.1432-1033.1977.tb11985.x
Summerton, J. E., Weller, D. D., 1991. Uncharged morpholino-based polymers having achiral intersubunit linkages. U.S. Pat. No. 5,034,506 A.
Summerton, J. E., Weller, D. D., Stirchak, E. P., 1993. Alpha-morpholino ribonucleoside derivatives and polymers thereof. U.S. Pat. No. 5,235,033 A.
Taylor, D. M., Morgan, H., D′ Silva, C., 1991. Characterization of chemisorbed monolayers by surface potential measurements. J. Phys. Appl. Phys. 24, 1443. doi:10.1088/0022-3727/24/8/032
Thompson, J. F., Steinmann, K. E., 2010. Single molecule sequencing with a HeliScope genetic analysis system. Curr. Protoc. Mol. Biol. Ed. Frederick M Ausubel Al Chapter 7, Unit 7.10. doi:10.1002/0471142727.mb0710s92
Tsavachidou, D., 2015. Methods for Nucleic Acid Base Determination. WO/2015/167972.
Tseung, K. K., Takayama, G., Rhett, N. K., Corl, M. V., 2004. Method for automated staining of specimen slides. U.S. Pat. No. 6,746,851 B1.
Ts'o, P. O. P., Miller, P. S., 1984. Nonionic nucleic acid alkyl and aryl phosphonates and processes for manufacture and use thereof. U.S. Pat. No. 4,469,863 A.
Turner, S., Korlach, J., 2013. Modified base detection with nanopore sequencing. US20130327644 A1.
von Kiedrowski, G., Wlotzka, B., Helbing, J., Matzen, M., Jordan, S., 1991. Parabolic Growth of a Self-Replicating Hexadeoxynucleotide Bearing a 3′-5′-Phosphoamidate Linkage. Angew. Chem. Int. Ed. Engl. 30, 423-426. doi:10.1002/anie.199104231
Walker, G. T., Little, M. C., Nadeau, J. G., Shank, D. D., 1992. Isothermal in vitro amplification of DNA by a restriction enzyme/DNA polymerase system. Proc. Natl. Acad. Sci. 89, 392-396.
Wang, H., Hays, J. B., 2000. Preparation of DNA substrates for in vitro mismatch repair. Mol. Biotechnol. 15, 97-104. doi:10.1385/MB:15:2:97
Williams, J., Anderson, J., Urlacher, T., Steffens, D., 2007. Mutant polymerases for sequencing and genotyping. US20070048748 A1.
Williams, P., Hayes, M. A., Rose, S. D., Bloom, L. B., Reha-Krantz, L. J., Pizziconi, V. B., 2006. Sequencing DNA using polymerase, fluorescence, chemiluminescence, thermopile, thermistor and refractive index measurements; microcalorimetric detection. U.S. Pat. No. 7,037,687 B2.
Xayaphoummine, A., Bucher, T., Isambert, H., 2005. Kinefold web server for RNA/DNA folding path and structure prediction including pseudoknots and knots. Nucleic Acids Res. 33, W605-610. doi:10.1093/nar/gki447
Xu, Y., Lunnen, K. D., Kong, H., 2001. Engineering a nicking endonuclease N. AlwI by domain swapping. Proc. Natl. Acad. Sci. 98, 12990-12995. doi:10.1073/pnas.241215698
Yau, E. K., 1997. Process for preparing phosphorothioate oligonucleotides. U.S. Pat. No. 5,644,048 A.
Zhu, Z., Samuelson, J. C., Zhou, J., Dore, A., Xu, S.-Y., 2004. Engineering strand-specific DNA nicking enzymes from the type IIS restriction endonucleases BsaI, BsmBI, and BsmAI. J. Mol. Biol. 337, 573-583. doi:10.1016/j.jmb.2004.02.003
Zuker, M., 2003. Mfold web server for nucleic acid folding and hybridization prediction. Nucleic Acids Res. 31, 3406-3415. doi:10.1093/nar/gkg595

What is claimed is:

1. A method of constructing consecutively connected copies of a nucleic acid molecule comprising two strands, first and second, said method applied to one or more nucleic acid molecules, and said method comprising the steps of:
(i) attaching a nucleic acid molecule comprising two strands to an adaptor comprising a nicking endonuclease recognition site, by ligating the 5′ end of the first strand and the 3′ end of the second strand of the nucleic acid molecule to the adaptor;
(ii) exposing the nucleic acid molecule and its surroundings to ligases to attach a hairpin adaptor not comprising a nicking endonuclease recognition site, to the 3′ end of the first strand and to the 5′ end of the second strand of the nucleic acid molecule;
(iii) exposing the nucleic acid molecule and its surroundings to nicking endonucleases recognizing said nicking endonuclease recognition site, thereby generating a nick with an extendable 3′ end: (a) in the first strand of the nucleic acid molecule whose 5′ end is ligated to the adaptor in step (i), or (b) in a segment of the adaptor ligated to the first strand of the nucleic acid molecule in step (i), or (c) between the adaptor and the first strand of the nucleic acid molecule whose 5′ end is ligated to the adaptor in step (i);
(iv) extending said extendable 3′ end by using polymerase molecules with strand displacing activity; and
(v) repeating steps (ii) through (iv) at least once, thereby allowing consecutive construction of copies of the nucleic acid molecule connected to one another.

2. A method of constructing consecutively connected copies of a nucleic acid molecule comprising two strands, said method applied to one or more nucleic acid molecules, and said method comprising the steps of:
(i) Ligating hairpin adaptors to a nucleic acid molecule, said hairpin adaptors comprising nicking endonuclease recognition sites;
(ii) Generating nicks with extendable 3′ ends within the nicking endonuclease recognition sites of said hairpin adaptors, by exposing the nucleic acid molecule and its surroundings to nicking endonucleases;
(iii) extending said extendable 3′ ends by using polymerase molecules with strand displacing activity, thereby generating hairpin constructs;
(iv) ligating hairpin adaptors to the hairpin constructs in step (iii), said hairpin adaptors not comprising said nicking endonuclease recognition sites, thereby generating circularized constructs comprising a single nicking endonuclease recognition site each;
(v) generating nicks with extendable 3′ ends within the nicking endonuclease recognition sites, by exposing to nicking endonucleases;
(vi) extending the 3′ ends of the nicks in step (v) by using strand-displacing polymerases;
(vii) repeating nick formation and extension, producing displaced single strands that can form hairpins with extendable 3′ ends;
(viii) extending the extendable 3′ ends from step (vii), thus producing long hairpin constructs;
(ix) ligating hairpin adaptors to the long hairpin constructs in step (viii), said hairpin adaptors regenerating nicking endonuclease recognition sites upon ligation; and
(x) repeating steps (v) through (ix).

3. The method according to claim 1, wherein all steps are conducted in the same reaction solution comprising nicking endonucleases, polymerases and ligases.

4. The method according to claim 3, wherein the reaction solution participates in temperature cycles comprising a temperature setting that favors nicking and extension and another temperature setting that favors ligation.

5. The method according to claim 1, wherein reagents used for at least two steps are included in a single reaction solution.
